(19) United States
(12) Patent Application Publication
(10) Pub. No.: US 2026/0224498 A1
(43) Pub. Date: Aug. 6, 2026

(54) COMPOSITION AND USE THEREOF

(71) Applicants: TOKUSHUKAI MEDICAL CORPORATION, Osaka-shi, Osaka (JP); StemMed Inc., Kobe-shi, Hyogo (JP); HUMAN LIFE CORD JAPAN INC., Tokyo (JP)

(72) Inventors: Amankeldi A. SALYBEKOV, Kamakura-shi, Kanagawa (JP); Shuzo KOBAYASHI, Kamakura-shi, Kanagawa (JP); Takayuki ASAHARA, Kamakura-shi, Kanagawa (JP)

(21) Appl. No.: 19/128,854

(22) PCT Filed: Oct. 6, 2023
(86) PCT No.: PCT/JP2023/036507

§ 371 Date: May 9, 2025

(30) Foreign Application Priority Data
Nov. 11, 2022 (JP) 2022-181301

(51) Int. Cl. A61K 9/50; A61P 13/12; C12N 15/113

(52) U.S. Cl. CPC A61K 9/5068; A61P 13/12; C12N 15/113; C12N 2310/141

(57) ABSTRACT

The present disclosure provides a cell-derived extracellular vesicle with reduced transplant rejection and having bioactivities against at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases. A composition for use in treating at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases includes an extracellular vesicle of a regenerative cell population induced from a biological sample-derived mononuclear cell or a culture thereof.

### TECHNICAL FIELD

The present disclosure relates to a composition and uses thereof.

### BACKGROUND ART

Cell-based treatments have been attempted for various diseases such as inflammatory diseases, kidney diseases, peripheral arterial diseases (PAD), cerebral infarction, and pulmonary hypertension (PHD). Autologous cells have been used in such treatments. However, when autologous cells are used, it is necessary to collect cells from the patient, process them, and then administer them back to the patient, which requires time and introduces variability in processing. For this reason, treatments using allogeneic cells have also been explored (Patent Literature 1).

### CITATION LIST

### Patent Literature

Patent Literature 1: JP 2011-529957A

### SUMMARY OF INVENTION

### Technical Problem

However, a problem arises when the allogeneic cells are transplanted into the patient: an immune response (transplant rejection) against the allogeneic cells is caused after the transplantation, the allogeneic cells are eliminated from the patient, and a sufficient therapeutic effect cannot be obtained.

Therefore, an object of the present disclosure is to provide a composition including a cell-derived extracellular vesicle that reduces transplant rejection and exhibits bioactivities against at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases.

### Solution to Problem

In order to achieve the above object, the present disclosure provides a composition for use in treating at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases. The composition includes an extracellular vesicle of a regenerative cell population induced from a biological sample-derived mononuclear cell or a culture thereof.

The present disclosure also provides a composition for use in treating at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, wherein the composition is the composition according to the present disclosure.

The present disclosure also provides a composition for use in promoting vascular endothelial cell proliferation, wherein the composition is the composition according to the present disclosure.

The present disclosure also provides a composition for use in inducing angiogenesis, wherein the composition is the composition according to the present disclosure.

The present disclosure also provides a composition for use in suppressing fibrosis, wherein the composition is the composition according to the present disclosure.

### Advantageous Effects of Invention

According to the present disclosure, it is possible to provide a composition including a cell-derived extracellular vesicle with reduced transplant rejection and having bioactivities against at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases.

### BRIEF DESCRIPTION OF DRAWINGS

FIGS. 1A to 1C are graphs showing the particle size, amount of production per cell, and mean diameter of extracellular vesicles in Example 1.

FIGS. 2A to 2D show marker expression profiles, an electron microscope image showing lipid bilayer structure of extracellular vesicles, and graphs showing secretion amount and protein amount of extracellular vesicles in Example 1.

FIGS. 3A and 3B are histograms showing the expression of markers in Example 1.

FIG. 4 is a graph showing the proliferation of HUVEC cells in Example 1.

FIGS. 5A and 5B are photographs and a graph showing the growth area of HUVEC cells in Example 1.

FIG. 6 is a graph showing the expression levels of miRNAs in Example 1.

FIGS. 7A and 7B are graphs showing the body weight and organ weight of rats in Example 1.

FIG. 8 is a set of graphs showing the proportions of CD3 positive cells, CD4 positive cells, CD8 positive cells, regulatory T cells, and CD 11b/c positive cells in Example 1.

FIG. 9 is a graph showing the expression level of miRNA in Example 1.

FIGS. 10A and 10B are graphs showing the serum creatinine values and BUN values in Example 1.

FIGS. 11A to 11F are photographs and graphs showing the degree of fibrosis in Example 1.

FIGS. 12A to 12D are photographs showing microvascular density in ischemic injured kidney tissue and graphs showing the number of CD31 positive microvessels and transcriptome analysis results in Example 1.

FIGS. 13A to 13D are photographs showing microvascular density in ischemic injured kidney tissue and graphs showing the number of CD31 positive microvessels on day 4 and day 28 after the onset of R-IRI in Example 1.

FIG. 14 is a photograph and a graph showing the results of IVIS measurement in Example 1.

FIGS. 15A to 15E are graphs and diagrams showing the results of various analytical methods used to elucidate the mechanism of fibrosis reduction, demonstrating that renal fibrosis is significantly reduced 4 weeks after the onset of K-IRI following administration of RACev in Example 1.

### DESCRIPTION OF EMBODIMENTS

### Definition

As used herein, the term “disease” includes at least one disease selected from the group consisting of kidney diseases, peripheral arterial diseases (PAD), pulmonary hypertension (PHD), spinal cord ischemia, non-occlusive mesenteric ischemia, ocular ischemic syndrome, fibrosis, and inflammatory diseases, and is not particularly limited as long as the disease can be treated with the composition of the present disclosure.

As used herein, the term “biological sample” refers to a sample derived from a living organism, from which constituent cells of the living organism can be extracted or isolated.

As used herein, the term “mononuclear cell” refers to a cell having a circular nucleus included in peripheral blood, bone marrow, umbilical cord blood, or the like. Examples of the mononuclear cell include lymphocytes, monocytes, macrophages, endothelial progenitor cells, and hematopoietic stem cells. The mononuclear cells may further include CD34 positive cells and/or CD133 positive cells.

As used herein, the term “extracellular vesicle” refers to a vesicle having a membrane secreted from a cell. An extracellular vesicle generally is believed to be formed within the endosome of the cell of origin and then released out of the cell. Thus, the extracellular vesicle typically includes a lipid bilayer membrane and a lumen, and the lumen is surrounded by the lipid bilayer membrane. The lipid bilayer includes lipid derived from the cell membrane of the cell of origin. The lumen includes a cytoplasm derived from the cell of origin. The extracellular vesicles are classified into, for example, exosome, micro vesicle (MV), apoptotic body, and the like based on their size and/or surface-marker.

As used herein, the term “regenerative cell population” refers to a cell population including cells having a regenerative function. The regenerative function means a function of repairing or improving the function of a tissue or an organ, and the regenerative cell population may have any one of these functions. As a specific example, the regenerative function may be vascular endothelial cell proliferation promoting activity and/or angiogenesis promoting activity.

As used herein, the term “cell population” refers to a cell preparation including desired cells or a composition including desired cells. Thus, in the following description, the cell population can also be referred to, for example, as a cell preparation or a composition. Within the cell population, the proportion of desired cells in all cells (hereinafter, also referred to as “purity”) can be quantified, for example, based on the proportion of desired cells expressing one or more markers. The purity refers, for example, to the proportion of desired cells among living cells. The purity can be measured using methods including, for example, flow cytometry, immunohistochemical methods, in situ hybridization, or the like.

In the following description, the purity of the cell preparation is, for example, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, or 99% or more.

As used herein, the term “culture” refers to cells obtained by culturing cells of a subject and/or a medium of the cells.

As used herein, the term “umbilical cord” refers to a white tubular tissue that connects the fetus and the placenta. In the present disclosure, the origin of the “umbilical cord” is not particularly limited, and examples thereof include mammals, for example, mice, rats, rabbits, dogs, cats, cows, horses, pigs, monkeys, dolphins, and sea lions. The origin of the “umbilical cord” is preferably a primate mammal, and more preferably a human.

As used herein, the term “umbilical cord blood” refers to fetus-derived blood contained in the umbilical cord.

As used herein, the term “miRNA” refers to a non-coding RNA having a base length of about 18 to about 25. The miRNA may also be referred to as “micro RNA”, “miR”, or the like.

As used herein, “miRNA precursor” means a transcript that originates from genomic DNA and contains non-coding RNA including one or more miRNA sequences. The miRNA precursor can also be referred to as “pri-miRNA” or “pre-miRNA”. The miRNA precursor usually has a hairpin structure and has a base sequence of a mature miRNA.

As used herein, the term “mir-X” refers to a miRNA precursor of number X, and the term “miR-X” refers to a mature form of miRNA of number X (miRNA). In the following description, when two miRNAs originate from opposing arms of the same miRNA precursor, the two miRNAs are indicated using the suffix “-3p” or “-5p”. Also, in the following description, when a miRNA precursor includes two miRNAs, the “miR-X” refers to miRNAs of both -3p and -5p, unless otherwise indicated. Also, in the following description, when the relative expression levels of two miRNAs are known, and the expression level of one miRNA is lower than that of the other miRNA, the miRNA having the lower expression level is indicated as the name with an asterisk. For the information of the base sequences of “mir-X” and “miR-X”, reference can be made to miRBase (http://www.mirbase.org/).

As used herein, the expression “promoting cell proliferation” means that cell proliferation of a subject is significantly promoted. The cell of a subject is a desired cell. The promoting can also be referred to as improving, enhancing, or increasing. As a specific example, “promoting cell proliferation” means that the cell proliferation of a subject is significantly promoted in a treatment group treated with a test substance as compared to that of a control group not treated with a test substance or that of a control group treated with a substance having no cell proliferation promoting activity. Therefore, in the following description, it can be said that “cell proliferation is promoted” if the cell proliferation of a subject is significantly promoted as compared to that of the control group, even if the cell proliferation activity in the control group is lowered as compared to that at the start of the test. The cell proliferation activity can be measured, for example, according to Example 1 described below.

As used herein, the expression “promoting angiogenesis” means that angiogenesis or vasculogenesis is significantly promoted. The promoting can also be referred to as improving, enhancing, or increasing. As a specific example, “promoting angiogenesis” means that the angiogenesis or vasculogenesis is significantly promoted in a treatment group treated with a test substance as compared to that of a control group not treated with a test substance or that of a control group treated with a substance having no angiogenesis promoting activity. Therefore, in the following description, it can be said that “angiogenesis is promoted” if the angiogenesis or the hyperplasia amount of the blood vessel is significantly promoted as compared to that of the control group, even if the angiogenesis or the vasculogenesis amount in the control group is lowered as compared to that at the start of the test. The angiogenesis promoting activity can be measured, for example, according to Example 1 described below.

As used herein, the expression “suppressing fibrosis” means that fibrosis of a subject is significantly suppressed. The term “suppressing” can also be referred to as reducing, lowering, stopping, inhibiting, or preventing. As a specific example, “suppressing fibrosis” means that the fibrosis of the subject is significantly suppressed in a treatment group treated with a test substance as compared to that of a control group not treated with a test substance or that of a control group treated with a substance having no fibrosis suppressing activity. Therefore, in the following description, it can be said that “fibrosis is suppressed” if fibrosis of the subject is significantly suppressed as compared to that of the control group, even if fibrosis is already suppressed in the control group as compared to that at the start of the test. The fibrosis suppressing activity can be measured, for example, according to Example 1 described below.

As used herein, the term “transplant rejection” or “immune rejection” refers to a host response that results in the elimination of transplants caused in a host by differences in major histocompatibility antigens during the transplantation of cells, tissues, or organs. The transplant rejection may also be referred to as “allotransplant rejection” in the case of allogeneic transplantation. The transplant rejection may also be referred to as “xenotransplant rejection” in the case of xenogeneic transplantation. The “transplant rejection” can be measured, for example, according to Example 1 described below.

As used herein, the expression “suppressing transplant rejection” or “suppressing immune rejection” means that transplant rejection or immune rejection is significantly suppressed. The term “suppressing” can also be referred to as reducing, lowering, stopping, inhibiting, or preventing. The expression “suppressing transplant rejection” means that the transplant rejection is significantly suppressed in a treatment group treated with a test substance as compared to that of a control group treated with a substance inducing the transplant rejection. As a specific example, in the case of examining the transplant rejection of the extracellular vesicles, when the transplant rejection is significantly suppressed in the treatment group of the extracellular vesicles as compared to that of the control group treated with cells from which the extracellular vesicles are derived, it can be said that the transplant rejection of the extracellular vesicles is suppressed. The “transplant rejection” can be measured, for example, according to Example 1 described below.

As used herein, the term “extraction” refers to a state in which a specific component is more concentrated than before extraction. Thus, the extraction can also be referred to as condensation or enrichment. The “extraction” can be carried out, for example, by performing at least one concentration process.

As used herein, the term “isolation” refers to a state in which a specific component is identified and separated and/or recovered from a component in its natural state. The “isolation” can be carried out, for example, by performing at least one purification process.

As used herein, the term “positive (+)” means that the signal detected by an analytical method such as flow cytometry of detecting using an antigen-antibody reaction is higher than that of a negative control reaction using a negative control cell that does not express the antigen or an antibody that does not react with the antigen.

As used herein, the term “negative (−)” means that the signal detected is equivalent to or lower than that of a negative control reaction using a negative control cell that does not express the antigen or an antibody that does not react with the antigen.

As used herein, the term “treatment” refers to therapeutic treatment and/or preventive treatment. As used herein, “treatment” means the treatment, cure, prevention, suppression, amelioration, or improvement of a disease, condition, or disorder, or stop, suppression, reduction, or delay of the progression of a disease, condition, or disorder. As used herein, “prevention” refers to a reduction in the likelihood of the onset of a disease or condition, or a delay in the onset of a disease or condition. The term “treatment” may be, for example, treatment for a patient who develops a target disease, or treatment of a model animal with a target disease.

As used herein, the term “effective dose” refers to an amount that, when administered to a patient with a disease, is sufficient to provide a therapeutic effect on the disease.

As used herein, the term “subject” refers to animals or cells, tissues, or organs derived from animals, and is used in a sense that includes, in particular, humans. The term animal means human and non-human animals. Examples of non-human animals include mammals such as mice, rats, rabbits, dogs, cats, cows, horses, pigs, monkeys, dolphins, and sea lions.

Hereinafter, the present disclosure will be described with reference to examples. However, the present disclosure is not limited to the following examples and the like, and can be changed and implemented at one's discretion. In addition, the descriptions of the respective embodiments can be referred to each other unless otherwise specified. In the following description, the expression “** to ** ” is used to include numerical values or physical values before and after “to”. Also, in the following description, the expression “A and/or B” includes “A only”, “B only”, and “both A and B”.

### Composition

In one aspect, the present disclosure provides a cell-derived extracellular vesicle with reduced transplant rejection and having bioactivities against at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, or a composition including the cell-derived extracellular vesicle. The composition of the present disclosure is a composition for use in treating at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, the composition including an extracellular vesicle of a regenerative cell population induced from a biological sample-derived mononuclear cell or a culture thereof.

Because the composition of the present disclosure has suppressed transplant rejection and is derived from the regenerative cell population, the composition of the present disclosure possesses bioactivities, such as vascular endothelial cell proliferation promoting activity and angiogenesis promoting activity, against at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases. Thus, the composition of the present disclosure can be suitably used, for example, for the treatment or prognosis improvement of at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, as described below.

The regenerative cell population is a cell population induced from mononuclear cells derived from the biological sample. The regenerative cell population includes, for example, cells having a regenerative function that can be induced from the mononuclear cells. Examples of the cell having a regenerative function include endothelial progenitor cells and anti-inflammatory macrophages, and may include Th2 cells and regulatory T cells (Treg). The endothelial progenitor cells and the anti-inflammatory macrophages have, for example, angiogenesis promoting activity and anti-inflammatory activity as the regenerative function, and thus are extremely useful in not only angiogenesis but also in suppression of inflammation at inflammatory sites such as ulcers. The regenerative cell population includes one or more types of cells having a regenerative function.

The endothelial progenitor cells (EPCs) are cells capable of differentiating into vascular endothelial cells. The endothelial progenitor cells can be identified using cell-surface markers, for example, CD34, CD133, KDR (kinase insert domain receptor, CD309), CD105, and the like.

The endothelial progenitor cells may be further classified according to the degree of differentiation. In this case, the endothelial progenitor cells can be further classified into differentiated EPC colony cells and undifferentiated EPC colony cells. The differentiated EPC colony cells mainly include cells having a diameter of 20 to 50 μm (CFU-large cell like EC, hereinafter also referred to as a “large EPC colony”). The undifferentiated EPC colony cells mainly include cells having a diameter of 20 μm or less (CFU-small cell like EC, hereinafter also referred to as a “small EPC colony”). As to the differentiation from the endothelial progenitor cells to vascular endothelial cells, it can be said that a small EPC colony that appears at an early stage is an EPC colony of early differentiation stage with excellent proliferative ability, while a large EPC colony that appears at a later stage is a colony of late differentiation stage with excellent angiogenic ability (see, e.g., Masuda H. et al., Circulation Research, 109:20-37 (2011), which is herein incorporated by reference). Since the differentiated EPC colony cells are, for example, superior to the undifferentiated EPC colony cells in regenerative function such as angiogenesis or repairing, the endothelial progenitor cells are preferably differentiated EPC colony-forming cells.

The anti-inflammatory macrophage is a type of macrophage that secretes anti-inflammatory cytokines (IL-10, IL-4) and the like, and is a cell that contributes to angiogenesis or repairing. The anti-inflammatory macrophage can be identified, for example, using cell-surface markers such as CD206, CD163, CD169, and the like. The anti-inflammatory macrophages are preferably M2 macrophages, for example, because they have an excellent regenerative function such as angiogenesis or repairing.

The proportion of the cells having a regenerative function in all cells of the regenerative cell population is not particularly limited, and is, for example, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, or 99% or more. The proportion of the cells can be calculated, for example, by measuring the proportion of intended cell-surface marker-positive cells using the cell-surface marker having a regenerative function. The measurement can be performed using, for example, flow cytometry.

The proportion of the cells having a regenerative function in all cells of the regenerative cell population is usually increased by a factor of two, preferably by a factor of four or more, as compared to, for example, the proportion of cells having a corresponding regenerative function in mononuclear cells derived from the biological sample.

When the cells having a regenerative function are the endothelial progenitor cells and the anti-inflammatory macrophages, the proportion of the cells having a regenerative function in all cells of the regenerative cell population can be calculated, for example, as the sum of the proportion of CD34 positive cells and the proportion of CD206 positive cells.

In the regenerative cell population, the cell having the regenerative function is induced from the mononuclear cell. Therefore, the biological sample used for inducing the regenerative cell population may be a biological sample containing the mononuclear cells. The mononuclear cells are rich in blood and lymph. Thus, the biological sample may be a tissue and/or an organ including blood vessels and/or lymphatic vessels, or may be blood and/or a lymphatic fluid. Specific examples of the biological sample include bone marrow; blood, for example, umbilical cord blood, peripheral blood, and the like; and lymph.

The biological sample may be a biological sample collected from a subject to be administered, treated, or medically cared with the composition of the present disclosure (hereinafter, collectively referred to as “subject to be administered”), or may be a biological sample collected from a subject other than the subject to be administered. From the standpoint of not being subject to limitations during preparation, it is desirable to use a biological sample collected from a subject other than the subject to be administered. The regenerative cell population may be cells (heterogeneous cells) derived from a biological sample collected from a subject other than a subject to be administered or cells (autologous cells) derived from a biological sample collected from a subject to be administered, as will be described in the Examples below. In the present disclosure, since the transplant rejection of the extracellular vesicles is suppressed, they can be suitably prepared even using a biological sample collected from a subject other than the recipient. In addition, it has been confirmed that the extracellular vesicles exert a therapeutic effect without being eliminated by, for example, immune rejection. Thus, the extracellular vesicles can be prepared, for example, as an off-the-shelf formulation.

The method for preparing the biological sample is not particularly limited, and can be appropriately determined according to, for example, the type of biological sample. As a specific example, when the biological sample is bone marrow, the biological sample can be obtained, for example, by performing bone marrow aspiration on the subject. When the biological sample is peripheral blood, the biological sample can be obtained by, for example, collecting venous blood. When the biological sample is umbilical cord blood, the biological sample can be obtained, for example, by isolating the umbilical cord and then collecting blood in the umbilical cord.

The method for separating mononuclear cells from the biological sample can be performed according to a method commonly performed in the art, for example, by separating the mononuclear cells and other components using a cell suspension prepared from the biological sample and a mononuclear cell separation solution such as Ficoll or Percoll.

The method for inducing the cells having the regenerative function from mononuclear cells derived from the biological sample can be performed by a known method according to, for example, the type of cells having a regenerative function. Specifically, when endothelial progenitor cells are induced from the mononuclear cells, the induction can be performed by culturing the mononuclear cells in the presence of, for example, SCF, IL-6, FLT3L, TPO, and/or VEGF. In addition, when anti-inflammatory macrophages are induced from the mononuclear cells, the induction can be performed by culturing the mononuclear cells in the presence of, for example, IL-10, IL-4, M-CSF and/or G-CSF.

The inducing method is preferably a method capable of inducing both the endothelial progenitor cells and the anti-inflammatory macrophages from the mononuclear cells. For the inducing method, for example, reference can be made to the inducing method described in WO 2014/051154, which is herein incorporated by reference.

Since the regenerative cell population can induce the cells having a regenerative function including, for example, the extracellular vesicles having angiogenic activity and anti-inflammatory activity, it is preferable that the mononuclear cells be cultured in the presence of factors such as a stem cell factor (SCF), interleukin-6 (IL-6), FMS like tyrosine kinase-3 ligand (Flt3L), thrombopoietin (TPO), and/or a vascular endothelial cell growth factor (VEGF). The culturing can be performed using a medium containing the factor.

As the mononuclear cells to be subjected to the culturing, a desired cell population obtained by sorting from mononuclear cells derived from the biological sample may be used, or mononuclear cells derived from the biological sample without being sorted may be used. As the mononuclear cells to be subjected to the culturing, it is preferable to use mononuclear cells (non-sorted mononuclear cells) obtained without sorting CD34 positive cells and/or CD133 positive cells. From this, the regenerative cell population obtained after the culturing can be, for example, a cell population in which the differentiated EPC colony-forming cells are increased (amplified), the anti-inflammatory macrophages are increased (amplified), and the anti-inflammatory Th2 cells and the regulatory T cells are increased (amplified), as compared to, for example, those of where mononuclear cells sorted as CD34 positive cells are used. Therefore, by using the non-sorted mononuclear cells, for example, a cell population in which the proportion of the cells having a regenerative function is enriched can be obtained as the regenerative cell population, thereby obtaining extracellular vesicles excellent in bioactivities, such as vascular endothelial cell proliferation promoting activity, angiogenesis promoting activity, fibrosis suppressing activity, transplant rejection suppressing activity, and immunosuppressive activity, against at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases.

The SCF is a glycoprotein of about 30,000 molecular weight consisting of 248 amino acids. Alternative splicing of the SCF results in either soluble SCF or membrane-bound SCF. The SCF for use in culturing can be either type of SCF, as long as it is useful for culturing a regenerative cell population such as EPC. The SCF is preferably soluble SCF. The origin or the like of SCF is not particularly limited, and is preferably a recombinant, particularly preferably a human recombinant because a stable feed is expected. The human recombinant SCF is commercially available. The concentration of SCF in the medium can be determined according to the type of SCF, and is not particularly limited as long as it is useful for culturing a regenerative cell population such as EPC. As a specific example, when the SCF is a human recombinant SCF, the concentration of SCF in the medium is, for example, 10 to 1000 ng/ml, preferably 50 to 500 ng/mL, and more preferably about 100 ng/mL.

The IL-6 is a glycoprotein having a molecular weight of about 21,000 isolated as a factor for inducing terminal differentiation of B cells into antibody-producing cells, and is known to be involved in immune responses, proliferation and differentiation of hematopoietic and nervous system cells, acute-phase reactions, and the like. The origin or the like of the IL-6 is not particularly limited, and is preferably a recombinant, particularly preferably a human recombinant because a stable feed is expected. The human recombinant IL-6 is commercially available. The concentration of IL-6 in the medium varies depending on the type of IL-6 used, and is not particularly limited as long as it is useful for culturing a regenerative cell population such as EPC. As a specific example, when the IL-6 is human recombinant IL-6, the concentration of IL-6 in the medium is, for example, 1 to 500 ng/ml, preferably 5 to 100 ng/ml, and more preferably about 20 ng/mL.

The Flt3L is known as a ligand for a receptor tyrosine kinase, which plays a key role in early hematopoietic control. As to the Flt3L, although several products that depend on alternative splicing are known, it has been reported that the Flt3L stimulates the proliferation of hematopoietic stem cells. The Flt3L may be any type of Flt3L as long as it is useful for culturing a regenerative cell population such as EPC. The origin or the like of Flt3L is not particularly limited, and is preferably a recombinant, particularly preferably a human recombinant because a stable feed is expected. The human recombinant Flt3L is commercially available. The concentration of Flt3L in the medium varies depending on the type of Flt3L used, and is not particularly limited as long as it is useful for culturing a regenerative cell population such as EPC. As a specific example, when the Flt3L is a human recombinant Flt3L, the concentration of Flt3L in the medium is, for example, 10 to 1000 ng/ml, preferably 50 to 500 ng/ml, and more preferably about 100 ng/mL.

The TPO is a type of hematopoietic cytokine and is known to specifically act in a process of producing megakaryocytes from hematopoietic stem cells, thereby promoting the production of megakaryocytes. The origin or the like of TPO is not particularly limited, and is preferably a recombinant, particularly preferably a human recombinant because a stable feed is expected. The human recombinant TPO is commercially available. The concentration of TPO in the medium varies depending on the type of TPO used, and is not particularly limited as long as it is useful for culturing a regenerative cell population such as EPC. As a specific example, when the TPO is a human recombinant TPO, the concentration of TPO in the medium is, for example, 1 to 500 ng/mL, preferably 5 to 100 ng/mL, and more preferably about 20 ng/mL.

The VEGF is a growth factor that acts specifically on EPC and is known to be produced primarily in perivascular cells. Alternative splicing of the VEGF results in several types of VEGF proteins of different sizes. The VEGF may be any type of VEGF as long as it is capable of colonizing EPC contained in the regenerative cell population, and is preferably VEGF165. The origin or the like of VEGF is not particularly limited, and is preferably a recombinant, particularly preferably a human recombinant because a stable feed is expected. The human recombinant VEGF is commercially available. The concentration of VEGF in the medium varies depending on the type of VEGF used, and is not particularly limited as long as it is useful for culturing a regenerative cell population such as EPC. When the VEGF is a human recombinant VEGF165, the concentration of VEGF in the medium is, for example, about 5 to 500 ng/ml, preferably about 20 to 100 ng/mL, and more preferably about 50 ng/mL.

As the various factors to be added to the medium, factors derived from animals of the same species as or different species from the animal from which the mononuclear cells are derived are used, and it is preferable to use factors derived from animals of the same species. By unifying the origin of the mononuclear cells and various factors, a regenerative cell population suitable for allogeneic transplantation such as allotransplantation, and extracellular vesicles derived therefrom are obtained. Furthermore, by using mononuclear cells derived from the subject to be administered, it is also possible to obtain extracellular vesicles suitable for isogeneic transplantation. Thus, by culturing a regenerative cell population containing EPC or the like in an environment that does not contain any components derived from a heterologous animal, the resulting regenerative cell population and the extracellular vesicles derived therefrom can reduce the risk of infection and rejection upon administration or the like.

The medium for use in the culturing may be prepared by dissolving the various factors in a basal medium so as to achieve a predetermined concentration or may be prepared by preparing a concentrate (stock solution) containing the various factors in advance and diluting the concentrate with a desired medium so as to achieve a predetermined concentration. The medium for use in the culturing may be prepared by dissolving various necessary factors in a commercially available basal medium so as to achieve a predetermined concentration, and then sterilizing by filter sterilization or the like, or may be prepared by aseptically adding a stock solution sterilized by filter sterilization or the like to a commercially available basal medium and then diluting. The filter sterilization can be carried out according to a method usually carried out in the art, for example, using a Millipore filter having a pore size of 0.22 μm or 0.45 μm.

As the basal medium, a basal medium commonly used in the art can be used. For example, a basal medium known as a medium for hematopoietic stem cell proliferation can be used. The basal medium may be a serum-containing medium or a medium containing no serum, i.e., a serum-free medium. In the culturing of the regenerative cell population, by using a serum-free medium as the medium and/or the basal medium, in the culturing (induction) of the regenerative cell population from the mononuclear cells, the cells having a regenerative function can be efficiently induced (amplified). Therefore, the medium is preferably a serum-free medium. Examples of the medium or the basal medium include DMEM, MEM, and IMDM.

The medium contains at least one factor selected from the group consisting of SCF, IL-6, FLT3L, TPO, and VEGF; preferably at least three factors selected from the group; or more preferably all factors in the group. Therefore, the medium for use in the culturing contains, for example, any of the following:
(1) SCF, (2) IL-6, (3) FLT3L, (4) TPO, or (5) VEGF;
(6) combination of SCF and IL-6, (7) combination of SCF and FLT3L, (8) combination of SCF and TPO, (9) combination of SCF and VEGF, (10) combination of IL-6 and FLT3L, (11) combination of IL-6 and TPO, (12) combination of IL-6 and VEGF, (13) combination of FLT3L and TPO, (14) combination of FLT3L and VEGF, or (15) combination of TPO and VEGF;
(16) combination of SCF, IL-6, and FLT3L, (17) combination of SCF, IL-6, and TPO, (18) combination of SCF, FLT3L, and TPO, (19) combination of SCF, FLT3L, and VEGF, (20) combination of IL-6, FLT3L, and TPO, or (21) combination of IL-6, FLT3L, and VEGF;
(22) combination of SCF, IL-6, FLT3L, and TPO, (23) combination of SCF, IL-6, FLT3L, and
VEGF, or (24) combination of IL-6, FLT3L, TPO and VEGF; or
(25) combination of SCF, IL-6, FLT3L, TPO, and VEGF.

More preferably, the medium contains SCF, IL-6, FLT3L, TPO, and VEGF. Still more preferably, the medium contains about 50 ng/mL VEGF, about 100 ng/mL SCF, about 20 ng/ml IL-6, 100 ng/mL FLT3L, and about 20 ng/ml TPO.

The culturing of the mononuclear cells using a medium containing the various factors can be carried out by adding a cell suspension containing the mononuclear cells to a medium containing the various factors. As the cell suspension, a body fluid (e.g., bone marrow fluid, umbilical cord blood, peripheral blood) containing the mononuclear cells may be used. The culturing conditions of the mononuclear cells are not particularly limited, and the culturing can be usually carried out under conditions performed in the art. As a specific example, the culturing is carried out at 37° C. for 7 days or more (for example, 10 days or more) in, for example, a 5% CO₂ atmosphere. The concentration (seeding density) of the mononuclear cells in the medium is not particularly limited as long as it allows culturing of a regenerative cell population such as EPC. The concentration (seeding density) of the mononuclear cells in the medium is, for example, about 0.5 to 10×10⁵ cells/mL, more preferably about 1 to 5×10⁵ cells/mL, and still more preferably about 3 to 4×10⁵ cells/mL.

The culture of the regenerative cell population may be a culture obtained in the induction of the regenerative cell population from the mononuclear cells or may be a culture obtained by culturing the induced regenerative cell population.

The medium and the culturing conditions of the regenerative cell population are not particularly limited, and for example, reference can be made to the description as to the medium and the culturing conditions of the mononuclear cells. As a specific example, as the medium for the regenerative cell population, for example, a medium obtained by adding serum, preferably human-derived serum, to the basal medium may be used. The origin of the serum may be the same as or different from the origin of the regenerative cell population, and is preferably the same as the origin of the regenerative cell population. The serum may contain the extracellular vesicles. Thus, when the serum is added to the basal medium, it is preferable to use the serum from which the extracellular vesicles have been removed. The serum from which the extracellular vesicles have been removed is commercially available. The concentration of the serum is, for example, from 1 to 20 (v/v) %, and preferably from 1 to 10 (v/v) %.

The culturing of the regenerative cell population can be carried out by adding a cell suspension containing the regenerative cell population to a medium of the regenerative cell population. The culturing conditions of the regenerative cell population are not particularly limited, and the culturing can be usually carried out under conditions performed in the art. As a specific example, the culturing is carried out at 37° C. for 1 to 10 days (for example, 1 to 2 days) in, for example, a 5% CO₂ atmosphere. The concentration (seeding density) of the regenerative cell population in the medium is not particularly limited as long as it allows culturing of the regenerative cell population. The concentration (seeding density) of the regenerative cell population in the medium is, for example, about 1×10⁴ to 1×10⁷ cells/mL, more preferably about 1×10⁵ to 1×10⁶ cells/mL, and still more preferably about 5×10⁵ cells/mL.

The extracellular vesicles are secreted from the cells of origin, as described above. Thus, the extracellular vesicles may be isolated or extracted from the regenerative cell population or a culture thereof.

The extracellular vesicles can be isolated or extracted from the regenerative cell population, for example, by disrupting the regenerative cell population, releasing the extracellular vesicles in the cells of the regenerative cell population, separating a soluble fraction from the obtained suspension, and then subjecting the obtained soluble fraction to a method for isolating or extracting. The method for isolating or extracting the extracellular vesicles can be performed according to a method commonly performed in the art, and can be performed, for example, by a method using centrifugation, an affinity purification method using a membrane protein derived from a regenerative cell population present in a membrane of the extracellular vesicles or using the membrane protein, or filtration using a porous membrane such as a filter. As the filter, for example, a commercially available membrane filter can be used, and as a specific example, a membrane filter having a pore size of 0.22 μm (manufactured by Millipore Corporation) can be used. The membrane protein of the extracellular vesicle is described below. The extracellular vesicle may be isolated or extracted by using a commercially available kit, and as a specific example, qEv isolation kit (manufactured by Izon Science Limited) can be used.

When the extracellular vesicles are isolated or extracted by the centrifugation, the centrifugation conditions may be any conditions capable of precipitating the extracellular vesicles, and for example, the centrifugation may be performed at 10000 to 300000×g or 90000 to 180000×g for 20 to 60 minutes. The temperature during the centrifugation is, for example, 0 to 10° C., preferably 0 to 4°C.

The culture is composed, for example, of the regenerative cell population and a medium (conditioned medium or culture supernatant) thereof. Thus, when the extracellular vesicles are isolated from the culture, the extracellular vesicles may be isolated or extracted from the regenerative cell population or the conditioned medium, or may be separated from both of the regenerative cell population and the conditioned medium. The method for separating the regenerative cell population and the conditioned medium from the culture can be performed according to a method commonly performed in the art, and can be performed by, for example, filter filtration, centrifugation, or the like. When the regenerative cell population is included, the extracellular vesicles can be isolated or extracted in the same manner as in the case of extracting or isolating the extracellular vesicles from the regenerative cell population described above.

When the conditioned medium is used, the method for isolating or extracting the extracellular vesicles can be performed, for example, by a method using centrifugation, an affinity purification method using a membrane protein derived from a regenerative cell population present in a membrane of the extracellular vesicles or using the membrane protein, or filtration using a porous membrane such as a filter. The extracellular vesicle may be isolated or extracted by using a commercially available kit, and as a specific example, qEv isolation kit (manufactured by Izon Science Limited) can be used.

Since the extracellular vesicles are derived from the regenerative cell population, the extracellular vesicles contain components derived from the regenerative cell population. The extracellular vesicle has, for example, a lipid bilayer membrane and a lumen, and the lumen is surrounded by the lipid bilayer membrane. In the extracellular vesicle, the lipid bilayer membrane is derived, for example, from the membrane of the regenerative cell population, specifically the membrane of the cell of the regenerative cell population. Examples of the membrane include cell membranes and membranes of intracellular organelles such as mitochondria, endoplasmic reticulum, endosome, lysosome, and Golgi apparatus. In the extracellular vesicle, the lumen has, for example, a cytoplasm derived from the regenerative cell population, specifically, a cytoplasm derived from cells of the regenerative cell population. Thus, the extracellular vesicles include, for example, miRNAs, DNAs, and/or proteins derived from the cytoplasm of the cells of the regenerative cell population.

The lipid bilayer membrane of the extracellular vesicle includes, for example, a membrane component of cells of the regenerative cell population. Thus, the extracellular vesicle can be defined, for example, using membrane components present in the regenerative cell population. The membrane component may be, for example, a membrane protein. Examples of the membrane protein include extracellular vesicle markers such as CD9, CD63, ALG-2-interacting protein X (Alix), heat shock protein 70 (Hsp-70), and the like; and cell-surface markers of the cells. The cell-surface marker can be appropriately determined according to the type of cell. When the regenerative cell population includes EPC, the cell-surface marker may be, for example, CD34, CD133, KDR (kinase insert domain receptor, CD309), CD105, or the like. When the regenerative cell population includes the anti-inflammatory macrophage, examples of the cell-surface marker include CD206, CD163, and CD169. The lipid bilayer membrane of the extracellular vesicle includes, for example, one or more membrane components. The membrane component can be measured by flow cytometry using, for example, an antibody against a molecule of a subject.

The lumen of the extracellular vesicle includes, for example, a cytoplasm of cells of the regenerative cell population. Thus, the extracellular vesicle can be defined, for example, using cytoplasmic components present in the regenerative cell population. The cytoplasmic component may be, for example, miRNA. Examples of the miRNA include miR-15b-5p, miR-29b-3p, miR-29c-3p, miR-92a-2-5p, miR-126-3p, miR-126-5p, miR-133a-3p, miR-133b, miR-146a-3p, miR-146b-5p, miR-150-5p, miR-181b-3p, miR-195-3p, miR-195-5p, miR-200b-5p, miR-302a-5p, miR-142-3p, miR-10a-3p, miR-17-3p, miR-20b-5p, miR-21-5p, miR-24-2-5p, miR-29a-5p, miR-30b-5p, miR-30c-5p, miR-30e-3p, miR-30e-5p, miR-32-5p, miR-92a-3p, miR-103a-2-5p, miR-144-5p, miR-148a-3p, miR-199b-5p, miR-200a-3p, miR-205-5p, miR-210-3p, miR-221-5p, miR-324-5p, miR-363-3p, miR-373-3p, miR-509-3p, miR-633, and let-7c-5p. Among the miRNAs, in particular, miR-92a-2-5p, miR-133a-3p, miR-133b, miR-146a-3p, miR-200b-5p, miR-302a-5p, and miR-142-3p are envisaged as specific miRNAs in the extracellular vesicles of the regenerative cell population. Thus, the lumen of the extracellular vesicle preferably includes miR-92a-2-5p, miR-133a-3p, miR-133b, miR-146a-3p, miR-200b-5p, miR-302a-5p, and/or miR-142-3p. The lumen of the extracellular vesicle includes, for example, one or more cytoplasmic components. The cytoplasmic component can be measured by flow cytometry using, for example, an antibody against a target molecule. When the cytoplasmic component is a nucleic acid molecule, the cytoplasmic component can be measured using, for example, a sequencer. In the case of a regenerative cell population derived from peripheral blood, the miRNA preferably includes, for example, miR-15b-5p, miR-29b-3p, miR-92a-2-5p, miR-146a-3p, miR-146b-5p, miR-150-5p, miR-195-3p, miR-195-5p, miR-200b-5p, miR-302a-5p, and/or miR-142-3p. In the case of a regenerative cell population derived from umbilical cord blood, the miRNA preferably includes, for example, miR-10a-3p, miR-17-3p, miR-20b-5p, miR-21-5p, miR-24-2-5p, miR-29a-5p, miR-30b-5p, miR-30c-5p, miR-30e-3p, miR-30e-5p, miR-32-5p, miR-92a-3p, miR-103a-2-5p, miR-144-5p, miR-148a-3p, miR-199b-5p, miR-200a-3p, miR-205-5p, miR-210-3p, miR-221-5p, miR-324-5p, miR-363-3p, miR-373-3p, miR-509-3p, miR-633, and/or let-7c-5p.

Among the miRNAs, miR-20b-5p, miR-30b-5p, miR-30c-5p, miR-30e-3p, miR-30e-5p, miR-32-5p, miR-92a-2-5p, miR-92a-3p, miR-199b-5p, miR-200a-3p, miR-363-3p, and miR-509-3p are generally known to have anti-apoptotic activity. Among the miRNAs, miR-17-3p, miR-20b-5p, miR-103a-2-5p, miR-150-5p, miR-181b-3p, miR-200a-3p, miR-205-5p, miR-302a-5p, miR-324-5p, miR-363-3p, and miR-633 are generally known to have cell proliferation promoting activity. Among the miRNAs, miR-150-5p is generally known to have cell migration promoting activity. Among the miRNAs, miR-21-5p, miR-29a-5p, miR-29b-3p, miR-29c-3p, miR-133a-3p, miR-133b, miR-200a-3p, and miR-373-3p are generally known to have anti-fibrotic activity, in particular, TGF-ß expression suppressing activity. Among the miRNAs, miR-15b-5p, miR-29c-3p, miR-126-3p, miR-126-5p, miR-146a-3p, miR-146b-5p, miR-181b-3p, miR-195-3p, miR-195-5p, miR-200b-5p, miR-210-3p, miR-221-5p, miR-633, and let-7c-5p are generally known to have angiogenesis promoting activity. Among the miRNAs, miR-10a-3p, miR-21-5p, miR-24-2-5p, miR-142-3p, miR-144-5p, and miR-148a-3p are generally known to have anti-inflammatory activity.

In the extracellular vesicle, a component contained in the lumen may be replaced with a component other than the cytoplasm of the regenerative cell population. In such a case, the lumen is loaded with a desired cargo. Examples of the cargo include a low molecular weight compound, a protein or a peptide such as an antibody, and a nucleic acid.

The mean diameter (weighted average) of the extracellular vesicles is, for example, 1 to 500 nm, preferably 10 to 250 nm, and more preferably 30 to 150 nm. The mean diameter can be measured in accordance with Example 1 described below. The mean diameter of the extracellular vesicles can be adjusted by, for example, filtering a liquid containing the extracellular vesicles using a filter having a desired pore size or the like.

The composition of the present disclosure may further include a pharmaceutically acceptable carrier. Examples of the carrier include suspensions, dissolve assists, stabilizers, isotonic agents, preservatives, adsorptions, surfactants, diluents, media, pH modifiers, painless agents, buffers, sulfur-containing reducing agents, and antioxidants for administration of the extracellular vesicles. The carrier can be suitably added to the extent that it does not interfere with the effectiveness of the present disclosure.

The composition of the present disclosure may be used, for example, in vitro or in vivo. The composition of the present disclosure can be used, for example, as a research reagent or as a pharmaceutical.

The subject to which the composition of the present disclosure is administered is not particularly limited. When the composition of the present disclosure is used in vivo, the subject to be administered may be, for example, a human or a non-human animal other than a human. Examples of the non-human animal include mammals such as mice, rats, rabbits, dogs, cats, cows, horses, pigs, monkeys, dolphins, and sea lions; birds; and fish. When the composition of the present disclosure is used in vitro, the subject to be administered may be, for example, a cell, a tissue, an organ, or the like, and the cell may be, for example, a cell collected from a living body, a cultured cell, or the like, and the tissue or organ may be, for example, a tissue (living tissue) or an organ collected from a living body. Examples of the cell include myocytes, induced pluripotent stem cells (iPS cells), and stem cells.

When the composition of the present disclosure is used in vivo, the subject to be administered may be, for example, a subject diagnosed with or suspected of having kidney disease. Examples of the kidney disease include acute kidney injury, transitional phase chronic kidney diseases, chronic kidney diseases, and immunosuppression after kidney transplantation.

When the composition of the present disclosure is used in vivo, the subject to be administered may be, for example, a subject diagnosed with or suspected of having peripheral arterial disease.

When the composition of the present disclosure is used in vivo, the subject to be administered may be, for example, a subject diagnosed with or suspected of having cerebral infarction.

When the composition of the present disclosure is used in vivo, the subject to be administered may be, for example, a subject diagnosed with or suspected of having pulmonary hypertension.

When the composition of the present disclosure is used in vivo, the subject to be administered may be, for example, a subject diagnosed with or suspected of having spinal cord ischemia.

When the composition of the present disclosure is used in vivo, the subject to be administered may be, for example, a subject diagnosed with or suspected of having non-occlusive mesenteric ischemia.

When the composition of the present disclosure is used in vivo, the subject to be administered may be, for example, a subject diagnosed with or suspected of having ocular ischemic syndrome.

When the composition of the present disclosure is used in vivo, the subject to be administered may be, for example, a subject diagnosed with or suspected of having fibrosis. Examples of the fibrosis include pulmonary fibrosis, myocardial fibrosis, and bone marrow fibrosis. Examples of the inflammatory disease include autoimmune diseases, inflammatory bowel diseases, and inflammatory muscle diseases.

When the composition of the present disclosure is used in vivo, the subject to be administered may be, for example, a subject diagnosed with or suspected of having inflammatory diseases. Examples of the inflammatory disease include autoimmune diseases, inflammatory bowel diseases, and inflammatory muscle diseases. Examples of the autoimmune disease include rheumatoid arthritis, systemic lupus erythematosus, polymyositis, Sjögren's syndrome, systemic sclerosis, glomerulonephritis, and vasculitis. Examples of the inflammatory bowel disease include ulcerative colitis and Crohn's disease. Examples of the inflammatory muscle disease include dermatomyositis, polymyositis, necrotizing autoimmune myositis, and (idiopathic) inclusion body myositis.

The use conditions (administration conditions) of the composition of the present disclosure are not particularly limited, and for example, the dosage form, the administration timing, the dosage, and the like can be appropriately set according to the type of subject to be administered, and the like.

Examples of the method for administering the composition of the present disclosure include oral administration and parenteral administration such as intravenous administration, and, for example, intravenous administration is preferable because it allows safe and stable administration regardless of the skill of the administrator.

The dosage of the composition of the present disclosure is an effective dose, and in particular, the dose that, when administered to a subject, can give a therapeutic effect on at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases as compared to a subject who has not been administered. Specifically, the dosage can be appropriately determined according to, for example, the age, weight, symptoms, and the like of the subject. Specifically, the dosage may be, for example, as the number of extracellular vesicles, 10⁴ to 10⁹ cells/kg weight, 10⁴ to 10⁸ cells/kg weight, or 10⁴ to 10⁷ cells/kg weight, and is preferably 10⁴ to 10⁸ cells/kg weight or 10⁴ to 10⁷ cells/kg weight per administration.

The number of administrations of the composition of the present disclosure is one or more than one, for example, such as 2 times, 3 times, 4 times, 5 times or more. The number of administrations may be determined as appropriate by monitoring the therapeutic effect on the subject. In the case of the plurality of administrations, the administration interval can be appropriately determined by monitoring the therapeutic effect on the subject, and the composition of the present disclosure can be administered once a day, once a week, once every two weeks, once a month, once every three months, or once every six months.

As described above, since the composition of the present disclosure has suppressed transplant rejection and is derived from the regenerative cell population, the composition of the present disclosure has bioactivities, such as angiogenesis promoting activity, against at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases. Thus, the composition of the present disclosure can be suitably used, for example, for the treatment or prognosis improvement of at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, as described below.

### Use of Extracellular Vesicle or Composition

In another aspect, the present disclosure provides a composition for use in treating at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, or a method of using the composition. In this case, the present disclosure provides a composition for use in treating at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, wherein the composition is the composition of the present disclosure. In addition, the present disclosure provides a method for treating a patient suffering from at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, the method including administering the composition of the present disclosure to the patient suffering from the diseases.

Examples of the disease include kidney disease, peripheral arterial disease (PAD), cerebral infarction, pulmonary hypertension (PHD), spinal cord ischemia, non-occlusive mesenteric ischemia, ocular ischemic syndrome, fibrosis, and inflammatory diseases. Examples of the kidney disease include acute kidney injury, transitional phase chronic kidney diseases, chronic kidney diseases, and immunosuppression after kidney transplantation. Examples of the fibrosis include pulmonary fibrosis, myocardial fibrosis, and bone marrow fibrosis. Examples of the inflammatory disease include autoimmune diseases, inflammatory bowel diseases, and inflammatory muscle diseases. Examples of the autoimmune disease include rheumatoid arthritis, systemic lupus erythematosus, polymyositis, Sjögren's syndrome, systemic sclerosis, glomerulonephritis, and vasculitis. Examples of the inflammatory bowel disease include ulcerative colitis and Crohn's disease. Examples of the inflammatory muscle disease include dermatomyositis, polymyositis, necrotizing autoimmune myositis, and (idiopathic) inclusion body myositis.

In another aspect, the present disclosure provides a composition for use in promoting vascular endothelial cell proliferation or a method using the composition. In this case, the present disclosure provides a composition for use in promoting vascular endothelial cell proliferation, wherein the composition is the composition of the present disclosure. The present disclosure also provides a method for promoting the vascular endothelial cell proliferation, including promoting the vascular endothelial cell proliferation by bringing the vascular endothelial cells into contact with a composition, wherein the composition includes the composition of the present disclosure.

In another aspect, the present disclosure provides a composition for use in inducing angiogenesis or a method using the composition. In this case, the present disclosure provides a composition for use in inducing angiogenesis, wherein the composition is the composition of the present disclosure. The present disclosure also provides a method for inducing angiogenesis, using the composition of the present disclosure.

In the method for inducing angiogenesis of the present disclosure, for example, the extracellular vesicle or the composition is administered to a subject to be induced with angiogenesis. The subject to be induced with angiogenesis is, for example, a patient with kidney disease, peripheral arterial disease (PAD), cerebral infarction, pulmonary hypertension (PHD), or the like. In this case, the induction of angiogenesis can also be referred to as induction of angiogenesis in the kidney, brain, lung, bone marrow, intestine, eye, ischemic muscle, or at a site with ischemia-reperfusion injury (e.g., central or peripheral blood vessel) of the patient.

In another aspect, the present disclosure provides a composition for use in suppressing fibrosis or a method using the composition. In this case, the present disclosure provides a composition for use in suppressing fibrosis, wherein the composition is the composition of the present disclosure. The present disclosure also provides a method for suppressing fibrosis, using the composition of the present disclosure.

In the method for suppressing fibrosis of the present disclosure, for example, the extracellular vesicle or the composition is administered to a subject for fibrosis suppression. The subject for fibrosis suppression may be, for example, a patient with kidney disease, peripheral arterial disease (PAD), cerebral infarction, pulmonary hypertension (PHD), or the like. In this case, the induction of angiogenesis can also be referred to as suppression of fibrosis in the kidney, brain, lung, bone marrow, intestine, eye, ischemic muscle, or at a site with ischemia-reperfusion injury (e.g., central or peripheral blood vessel) of the patient.

### Use of Extracellular Vesicle or Composition

The present disclosure provides a composition for use in treating at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases. The present disclosure provides a composition for use in promoting vascular endothelial cell proliferation.

The present disclosure provides a composition for use in promoting angiogenesis. The present disclosure provides a composition for use in suppressing fibrosis.

### EXAMPLES

Hereinafter, the present disclosure will be described in detail with reference to examples. It is to be noted, however, that the present disclosure is not limited to the aspect described in the examples. Unless otherwise indicated, commercially available reagents and kits were used according to the protocol.

### Example 1

The composition of the present disclosure was examined and found to have a vascular endothelial cell proliferation activity, to suppress transplant rejection, to restore a renal function, to suppress fibrosis, and to promote angiogenesis. In addition, the therapeutic effects of RAC-derived extracellular vesicles (RACev) on kidney ischemia-reperfusion injury (IRI) were evaluated in comparison with a vehicle control group.

### (1) Cell Culture

Human peripheral blood mononuclear cells (PBMCs) were isolated from healthy human volunteers after written informed consent was obtained according to the ethics committee (CS-00201623). Peripheral blood (30 mL) was collected using a vacutainer (manufactured by BD).

Isolation of peripheral blood mononuclear cells (PBMNC) from peripheral blood was performed by density gradient centrifugation using Histopaque-1077 (Sigma-Aldrich, Inc., #10771) according to the methods described in Asahara et al., Science, 275:964-7 (1997) and Amankeldi et. al., PLOSONE, (2019), 14(3): e0205477, which are herein incorporated by reference. The isolated PBMNC was washed with phosphate buffer (PBS)-ethylenediaminetetraacetic acid (EDTA) and then suspended in a serum-free medium (QQ medium) described below, thereby preparing a cell suspension. In the isolated PBMNC, the CD34 positive rate was 0.23±0.03%, and the CD133 positive rate was 0.20±0.07%. The number of CD34 positive cells per peripheral blood (100 mL) was (27.8±4.5)×104 and the number of CD133 positive cells per peripheral blood (100 mL) was (23.2±6.9)×104.

The serum-free medium (hereinafter also referred to as “QQ medium” or “QQ cm”) for use in culturing (inducing) the regenerative cell population was aseptically added to STEMLINE™ II Hematopoietic Stem Cell Expansion Medium (Sigma-Aldrich, Inc., Cat No. S0192) such that the following factors had predetermined concentrations.

### Concentrations of Factors in QQ Medium

rhSCF (manufactured by Peprotech, Inc., Cat. No. #300-07): 100 ng/ml
rhFlt3L (manufactured by Peprotech, Inc., Cat. No. #300-19): 100 ng/ml
rhTPO (manufactured by Peprotech, Inc., Cat. No. #300-18): 20 ng/ml
rhVEGF (manufactured by Peprotech, Inc., Cat. No. #100-02): 50 ng/ml
rhIL-6 (manufactured by Peprotech, Inc., Cat. No. #200-06): 20 ng/ml
penicillin/streptomycin (manufactured by Gibco), Cat. No. 15140122:100 U/100 μg/mL
r: recombinant
h: human origin

The PBMNC was seeded in a 10-cm dish (manufactured by Sumitomo Bakelite Co., Ltd.) so as to achieve 10×10⁷ cells/ 10 mL/dish and then cultured for 7 days at 37° C. and 5% CO₂ in a humid environment. From this, a regenerative cell population (RAC) was prepared. In the following, unless otherwise specified, the culturing conditions were at 37° C. and 5% CO₂ in a humid environment.

### (2) Preparation of Extracellular Vesicles

After the culturing, the RAC was recovered and then centrifuged. The obtained pellet was suspended in an extracellular vesicle production medium so as to prepare a cell suspension. The extracellular vesicle production medium was composed of X-vivo 15 Media (manufactured by Lonza, Cat. No. BE02-054Q) containing 5% (v/v) of exosome-removed fetal calf sera. The resulting cell suspension was seeded in a 10-cm dish so as to achieve 2.5×10⁵ cells/mL/dish (250 k) or 5×10⁵ cells/mL/dish (500 k), and then cultured for 48 hours.

After the culturing, the culture supernatant (conditioned medium) was recovered, and then the culture supernatant was centrifuged at 300×g for 10 minutes at 4° C. to remove the cells. The resulting supernatant fraction was then centrifuged at 2000×g for 20 minutes at 12° C. and then the supernatant fraction was recovered to remove apoptotic vesicles and microvesicles. The supernatant fraction was filtered through a 0.2-μm filter (manufactured by Millipore Merck, Cat. No. SLGS033SS) to remove extracellular vesicles having a particle size exceeding 200 nm. Then, the obtained filtrate was centrifuged at 174000×g for 110 minutes at 4° C. to precipitate extracellular vesicles (exosomes) to recover. The recovered precipitate was suspended in the medium or buffer (up to 3×50 μ) for use in each experiment described below. The number and size of EVs were then measured by nanoparticle tracking analysis using NanoSight 500 (Malvern Panalytical) according to the manufacturer's manual.

In order to label the extracellular vesicles, after the 0.2-μm filter filtration, CM-Dil dye (C7000, manufactured by Thermo Fisher Scientific Inc.) was added to the filtrate so as to achieve 2 umol/l. Then, the obtained reactant was centrifuged at 174000×g for 110 minutes at 4° C., and then the extracellular vesicles were washed with PBS and centrifuged under the same conditions. The washing and centrifugation were performed in the same manner again. The recovered precipitate was then suspended in the medium or buffer (up to 3×50 μl) for use in each experiment described below.

The labeled extracellular vesicles and control solution (saline) were injected through the tail vein using a 24G vascular catheter one day after the onset of R-IRI. Three hours after extracellular vesicle injection, the subject animals were euthanized and organs including kidney, spleen, lung, and heart were removed for further extracellular vesicle tracking analysis. The constriction of extracellular vesicles at the injury site was analyzed using IVIS Lumina III (manufactured by Perkin Elmer). The excitation wavelength filter of the Dil dye was set to 520 nm and the emission wavelength filter was set to 570 nm.

### (3) Animals Used in Experiments

All animal experimentation was conducted in accordance with the AAALAC International standards at Shonan Health Innovation Park (Approval No. AU-00030960). Male Lewis rats (6 to 10 weeks old, body weight: 150 to 250 g) were purchased from Charles River Laboratories through Oriental Yeast Co., Ltd. The animals used in the experiments were maintained under standard conditions (20 2° C., 50 to 60% relative humidity, 12 h/ 12 h light/dark cycle). The animals used in the experiment were allowed free access to water and food.

### (4) Characterization of Extracellular Vesicles

The extracellular vesicles prepared in “(2) Preparation of Extracellular Vesicles” were examined for their mean diameter, amount of production per cell, expression of markers, and structure of lipid membranes and lumens. Specifically, the mean diameter, the amount of production per cell, and the expression of markers were measured by flow cytometry. As to the flow cytometer, DxFlex Flow Cytometry (manufactured by Beckman Coulter, Inc.) with three lasers (405 nm, 488 nm, and 638 nm) was used after changing its configuration as follows. The 405/10 VSSC filter was moved to V 450 channel in WDM. Then, the configuration of the detector in the software (CytExpert software) was updated and assigned to VSSC channels in WDM. The flow path of the flow cytometer was washed with a washing solution (FlowClean) and then filtered through a 10-μm filter.

The flow cytometer (DxFlex FACS machine, manufactured by Beckman Coulter, Inc.) was calibrated using latex beads with different sizes (100 nm or 200 nm), and then the extracellular vesicles were measured. Further, with respect to the intensity of the obtained side scattered light (SSC), the particle size of each extracellular vesicle was calculated by comparing the intensity of the latex beads with the intensity of each extracellular vesicle. Then, the mean diameter (weighted average) was calculated based on the obtained particle size. In addition, the particle sizes of the extracellular vesicles were calculated, and the mean diameter (weighted average) was calculated in the same manner except that a nanoparticle tracking analyzer (NTA) (ZetaView®, manufactured by DKSH Holding Ltd.) was used instead of the flow cytometer. Then, it was verified that the mean diameter obtained by the flow cytometer and the mean diameter obtained by NTA were substantially the same.

The expression of the markers was measured using the flow cytometer after staining the extracellular vesicles with an anti CD9 antibody (PE labeling, manufactured by Biolegend Inc., Cat. No. #312106) or an anti CD63 antibody (PE labeling, manufactured by Biolegend Inc., Cat. No. #353004). As a control, the above procedure was conducted in the same manner except that the control antibody was used instead of the various antibodies.

As to the amount of production per cell, the amount of production of extracellular vesicles (the number of extracellular vesicles) per 2.5×10⁵ cells was calculated based on the counts in the flow cytometer and the measured sample volume.

The structure of the lipid membrane and the lumen were observed using a scanning electron microscope (JEM-1400, manufactured by JEOL Ltd.).

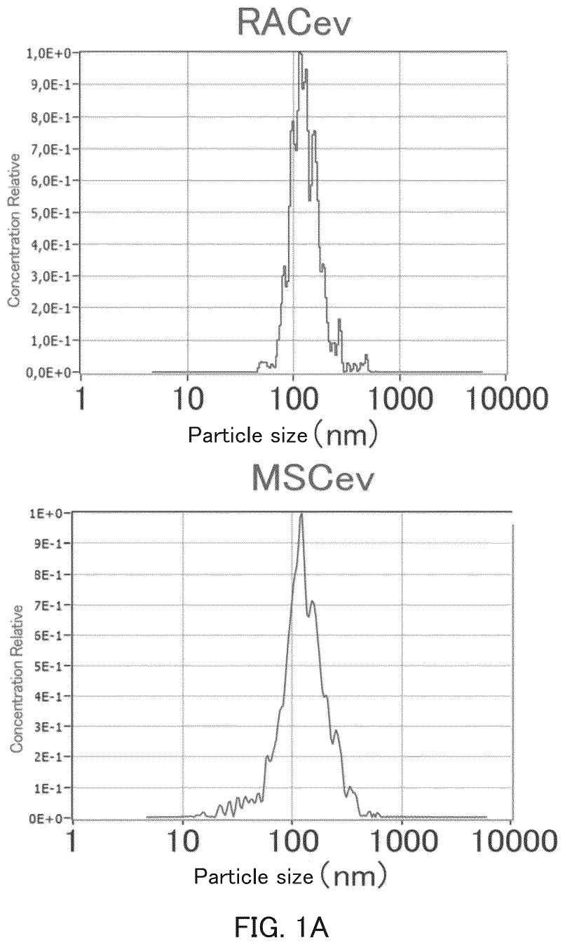

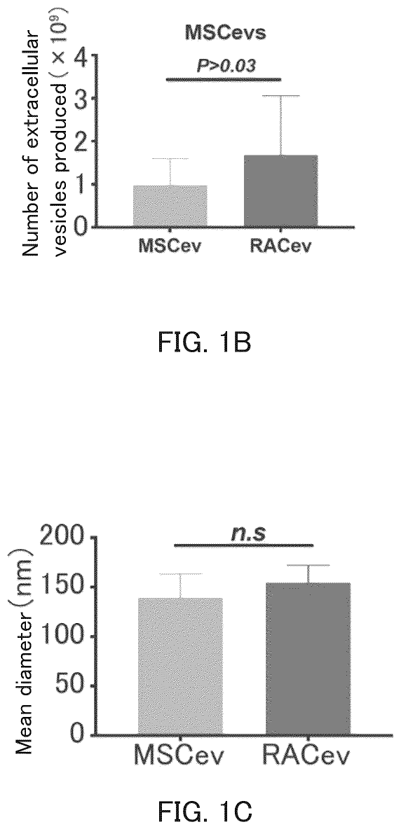

As a reference, the above procedure was conducted in the same manner except that human-derived mesenchymal stem cells (Cat. No. BE02-054Q, manufactured by MSC, Lonza) were used instead of the RAC. These results are shown in FIGS. 1A to 4. The statistical processing in the graph was performed using a Mann-Whitney test.

### (5) Vascular Endothelial Cell Proliferation Promoting Activity

The vascular endothelial cell proliferation promoting activity of RACev was examined using human fetal-derived vascular endothelial cells (HUVEC, manufactured by Lonza, Cat. No. C2517A, Batch #. 18TL061650). Specifically, HUVEC cells were labeled by recovering HUVEC that had been subcultured 3 to 4 times, adding Cytopainter dye (manufactured by abcam, Cat. No. #ab176735) and incubating at 37° C. for 10 to 30 minutes. After the labeling, centrifugation and washing were repeated twice. The obtained HUVEC cells were seeded in 3.5-cm dish so as to achieve 5×10⁴ cells/2 mL/dish. The medium for subclturing HUVEC cells was EGM-2 (manufactured by Lonza), and the medium for measuring the HUVEC cell proliferation activity was EBM-2 (manufactured by Lonza). Extracellular vesicles (RACev or MSCev) isolated from RAC or MSC of 250 k or 500 k were then added to the respective dishes. Then, the cells were cultured under the above-described culturing conditions for 2 days. After the culturing, the dishes were observed using a phase contrast microscope (6-well dish, manufactured by Falcon). In the obtained retardation image, the number of cells per single high power field (HPF) was counted, and the proportion of the area occupied by HUVEC cells to the entire area was calculated as the growth area.

Further, after the culturing, HUVEC cells were recovered from the dishes, and the division of HUVEC cells was measured using a flow cytometer (FACS Verse, manufactured by BD Biosciences Co., Ltd.) with the label as an indicator. As a control, the above procedure was conducted in the same manner except that no RACev or MSCev were added.

### (6) Library Preparation, Sequencing, and Bioinformatics Analysis

In addition, RACev and MSCev added during culturing of HUVEC cells were analyzed for miRNA. Specifically, as to RACev and MSCev, miRNA was purified using a miRNA purification kit (miRNeasy mini kit and RNeasy MinElute Cleanup Kit, manufactured by QIAGEN). A library for sequencing was prepared using the obtained miRNA and a library construction kit (QIAseq™). The quality-testing of the libraries was performed using Agilent Bioanalyze and DNA chips (High Sensitivity DNA chip, manufactured by Agilent Technologies, Inc.). The sequencing of the library was decoded as a 76 bp single-ended read using the next generation sequencer (NextSeq 500, manufactured by Illumina, Inc.). The expression levels of the respective miRNAs (miR-181b-3p, miR-150-5p, miR-302a-5p, and miR-92a-2-5p) were calculated from the obtained sequence data. Relative expression levels were calculated based on the expression levels of the respective miRNAs in HUVEC cells to which no extracellular vesicles were added.

As a control, the above procedure was conducted in the same manner except that no extracellular vesicles were added. As the statistical processing in the graph, Tukey's multiple comparison test was performed after Two-way ANOVA test.

### (7) Induction of Kidney Ischemia-Reperfusion Injury

The rats were anesthetized with 3 to 4% sevoflurane (manufactured by Maruishi Pharmaceutical Co., Ltd.). Through a midline abdominal incision, the two renal pedicles were brought together and then fixed for 45 minutes using a microaneurysm clamp (Fine Science Tools (FST)). During the ischemic period, the rats were placed on a 37° C. thermal pad to maintain the body temperature. After removing the clamp, renal blood flow was examined for 1 minute, and after confirming that the kidneys had returned to their original color, the abdomen was closed. Sham-operated rats underwent the same surgical procedure except that the microaneurysm clamp was not applied.

### (8) Confirmation of Transplant Rejection

One day after the onset of kidney ischemia-reperfusion injury (K-IRI), using a 24G vascular catheter (manufactured by Terumo), RACev (1×10⁶ extracellular vesicles derived from regeneration-associated cells) was transplanted to the treatment group, and PBS was transplanted to the control group. The transplantation was performed via the tail vein as described above. At the end of each experiment, animals were euthanized by overdosing with 5% sevoflurane, and after confirming euthanasia, vital organs such as kidneys, lungs, spleens, and livers were removed.

Specifically, the extracellular vesicles (RACev or MSCev) isolated from RAC or MSC of 500 k were diluted with PBS and the resulting dilution was administered from the tail vein at predetermined times (after 30 minutes, 1 day, and 2 days) after inducing the ischemia-reperfusion injury. The first body weight measurement was then performed before the onset of the ischemia-reperfusion injury (baseline), and then the body weight was measured once a week until 4 weeks after the onset of the injury. Kidneys were collected and weighed 4 days and 4 weeks after the onset. In addition, peripheral blood and kidney were collected 4 weeks after the onset. The proportions of CD3 positive cells, CD4 positive cells, CD8 positive cells, regulatory T cells, and CD11b/c positive cells in the peripheral blood were measured using a flow cytometer. As a control, the above procedure was conducted in the same manner except that no RACev or MSCev was administered. As to RACev and MSCev administered, the relative expression level of the respective miRNA (miR-142-3p) was calculated. Dunn's multiple comparison test was performed as the statistical processing in the graph.

### (9) Immunohistochemical Analysis

Systemic perfusion was performed with heparinized PBS (1000 U/500 mL), and the kidneys were fixed with 4% paraformaldehyde (manufactured by FUJIFILM Corporation, Cat.

No. #163-20145) overnight, embedded in paraffin, and cut into sections having a 5 to 3 mm thickness. Sections were stained with PAS (Periodic acid-Schiff) and with Masson's trichrome to determine the extent of fibrosis. To analyze microvessel density, sections were cultured with anti-CD31 (1:100) antibody and DAB. All tissue photographs were taken at a microscope magnification of 200× using an all-in-one fluorescence microscope BZ-X 800 (manufactured by KEYENCE CORPORATION.). Histological data were evaluated using Keyence Analyzer (manufactured by Keyence) in 3 to 5 high-power fields per section.

The original FASTQ files generated by the NextSeq were uploaded to the Qiagen GeneGlobe Data Analysis Center (https://geneglobe.qiagen.com) and aligned to miRBase v21 (http://www.mirbase. org). All reads assigned to specific miRs were counted, associated UMIs were tallied, and unique molecules were counted. The miR UMI count foundation was subjected to downstream analysis using StrandNGS 3.4 software (manufactured by Agilent Technologies). UMI counts were quantified using the trimmed mean M-value (TMM) method. To determine target genes of miRs with different expression levels, all information from PITA miRBase version 18, microRNA. org miRBase version 18, and TargetScan version 6.0 was integrated.

Next, gene ontology (GO) analysis and pathway statistical analysis were performed to determine the biological function of the target genes. The pathway statistical analysis was performed on the pathway collection of the Wiki Pathways database using the Path Visio tool to determine pathways considering the number of genes and target genes included in the pathways.

The kidney tissue was harvested 4 to 6 days after the onset of R-IRI and total RNA was isolated using a nucleic acid extraction system according to the manufacturer's instructions. RNA integrity scores were checked using an Agilent Bioanalyzer (Agilent Technologies, Santa Clara, California, USA).

### (10) Statistical Analysis

All values are expressed as mean value±SEM. The Mann-Whitney U test was used for two and three nonparametric groups, followed by Dunn's multiple comparison test. One-way analysis of variance was applied to three parametric groups, followed by Dunn's multiple comparison test or Tukey's multiple comparison test. Two-way analysis of variance was applied for multiple comparisons between groups at different time points, followed by Tukey's post-test. Differentially expressed miRs were determined using an absolute threshold of fold change ≥2.

All statistical analyses were performed using GraphPad Prism 9.1 (manufactured by GraphPad Software, Inc.). Statistical significance was set at P<0.05.

### Results

### (1) Characterization of Extracellular Vesicles

FIGS. 1A to 1C are graphs showing the particle size, amount of production per cell, and mean diameter of extracellular vesicles. FIG. 1A shows the result of the particle size, FIG. 1B shows the result of the production amount, and FIG. 1C shows the result of the mean diameter.

In FIG. 1A, the horizontal axis indicates the particle size (logarithm), and the vertical axis indicates the relative number. In FIG. 1B, the horizontal axis indicates the type of sample, and the vertical axis indicates the number of extracellular vesicles produced. In FIG. 1C, the horizontal axis indicates the type of sample, and the vertical axis indicates the mean diameter. As shown in FIGS. 1A and 1C, RAC-derived extracellular vesicles (RACev) were slightly larger than MSC-derived extracellular vesicles (MSCev), and the mean diameter of RACev was 145 nm and the mean diameter of MSCev was 130 nm. In addition, as shown in FIG. 1B, RAC produced significantly more extracellular vesicles per cell than MSC.

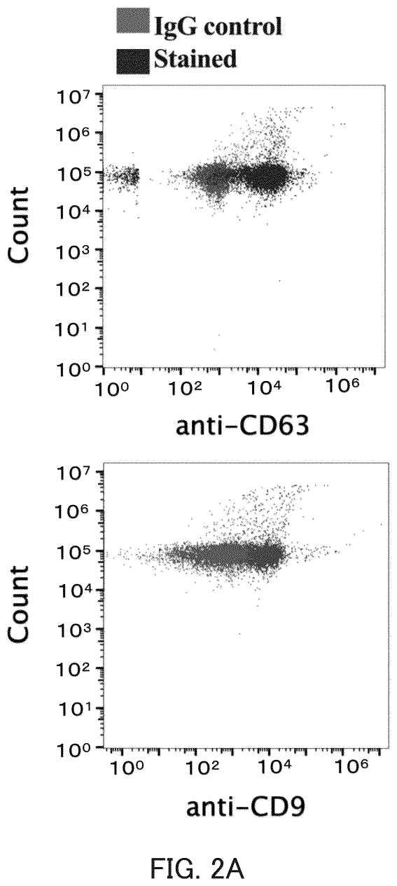

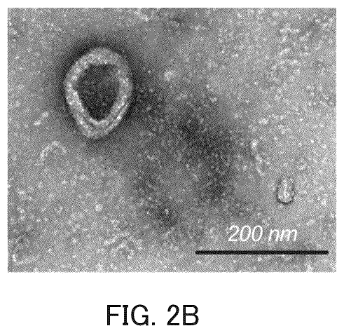

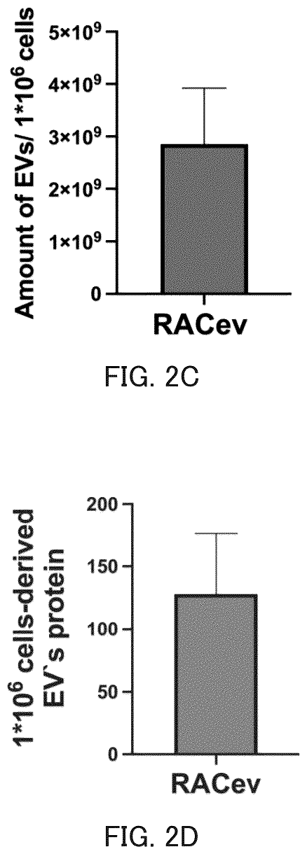

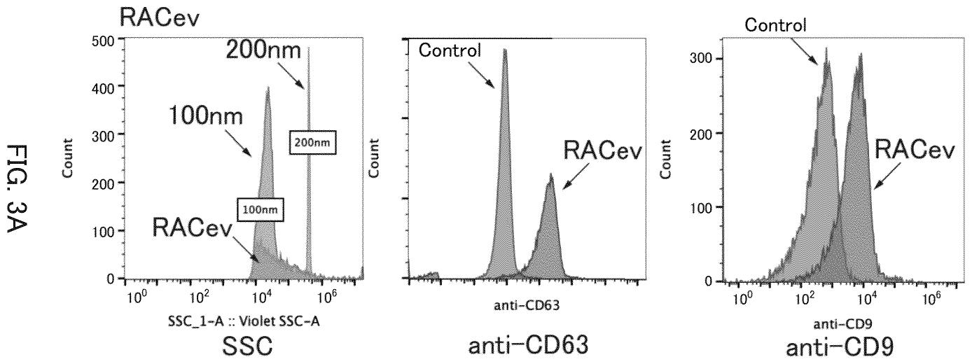

FIGS. 2A to 2D show marker expression profiles, an electron microscope image showing lipid bilayer structure of extracellular vesicles, and graphs showing secretion amount and protein amount of extracellular vesicles. FIG. 2A shows the results of anti-CD9 and anti-CD63 EV biomarker expression profiles in RACev, FIG. 2B shows a transmission electron microscope image showing the lipid bilayer structure of extracellular vesicles, FIG. 2C shows the quantification results of extracellular vesicle secretion amount per 1 million RACs, and FIG. 2D shows the number of all proteins in RACev derived from 1 million RACs. In FIG. 2A, the horizontal axis indicates the expression level of anti-CD63 or anti-CD9 positive RACev by flow cytometry, and the vertical axis indicates the size of the IgG control and the stained RACev. In FIG. 2C, the horizontal axis indicates the type of sample, and the vertical axis indicates the amount of extracellular vesicle secretion. In FIG. 2D, the horizontal axis indicates the type of sample and the vertical axis indicates the number of all proteins in RACev derived from RACs. As shown in FIG. 2A, the presence of extracellular vesicle-specific markers (CD9, CD63) was observed. As shown in FIG. 2B, a lipid bilayer structure was observed. Furthermore, as shown in FIGS. 2C and 2D, the average number of extracellular vesicles (RACev) obtained from 1 million RACs and the number of all proteins in RACev were 2.89×109±4.7×108 and 128±21.5, respectively. The size of the RACev was 154.5±6.7 nm.

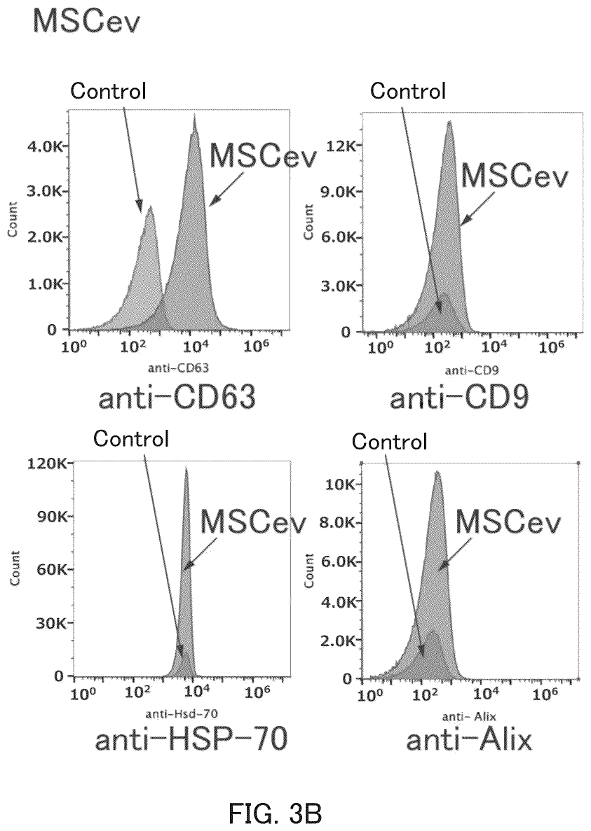

FIGS. 3A and 3B are histograms showing the expression of markers. FIG. 3A shows the results of RACev and FIG. 3B shows the results of MSCev. In FIGS. 3A and 3B, the horizontal axis indicates the SSC intensity or fluorescent intensity of each marker, and the vertical axis indicates the count. As shown in the left graph of FIG. 3A, it was found that the particle size of extracellular vesicle can be measured by a flow cytometer by using latex beads having known particle sizes. As shown in the middle and right graphs of FIG. 3A, the expressions of CD63 and CD9 were observed in RACev. As shown in FIG. 3A and B, the expression level of CD63 was higher in RACev than that of in MSCev.

These results show that RACev meets MISEV2018 (Thery C et. al., 2018, J. Extracell Vesicles 7:1535750, which is herein incorporated by reference), and therefore is found to be an extracellular vesicle.

### (2) Vascular Endothelial Cell Proliferation Promoting Activity

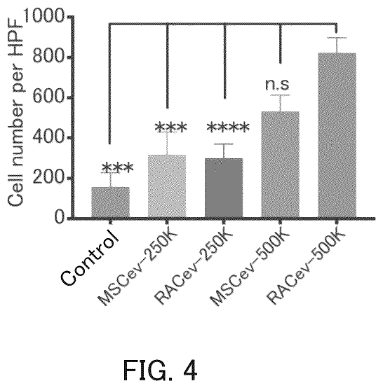

FIG. 4 is a graph showing the HUVEC cell proliferation. In FIG. 4, the horizontal axis indicates the type of sample, and the vertical axis indicates the number of cells per single high power field (HPF). As shown in FIG. 4, RACev and MSCev promoted the vascular endothelial cell proliferation as compared to that of the control. Also, as shown in FIG. 4, RACev significantly promoted the vascular endothelial cell proliferation as compared to that of MSCev (MSCev-250K vs. RACev-250K: P>0.001, MSCev-500K vs. RACev-500K: P>0.0001).

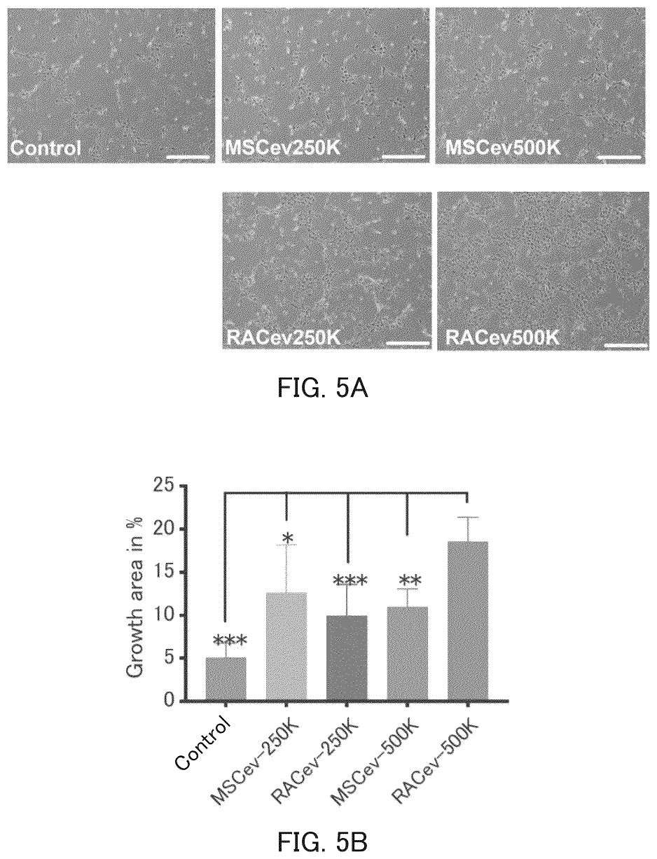

Next, FIGS. 5A and 5B are photographs and a graph showing the growth area of HUVEC cell. FIG. 5A shows phase difference images, and FIG. 5B shows a graph of the growth area. In FIG. 5B, the horizontal axis indicates the type of sample, and the vertical axis indicates the growth area. As shown in FIGS. 5A and 5B, RACev and MSCev had an increased growth area of vascular endothelial cells as compared to that of the control. Also, as shown in FIGS. 5A and 5B, RACev significantly increased the growth area of vascular endothelial cells as compared to that of MSCev (MSCev-500K vs. RACev-500K: P>0.0007). Incidentally, although it is not shown, during observation of the HUVEC cell proliferation over time using labeled RACev, it was observed that when the HUVEC cells incorporate RACev, the HUVEC cells proceeded to the S phase. It was also verified that the intravascular cell proliferation promoting activity by RACev was maintained up to the eighth generation, i.e., maintained during eight divisions.

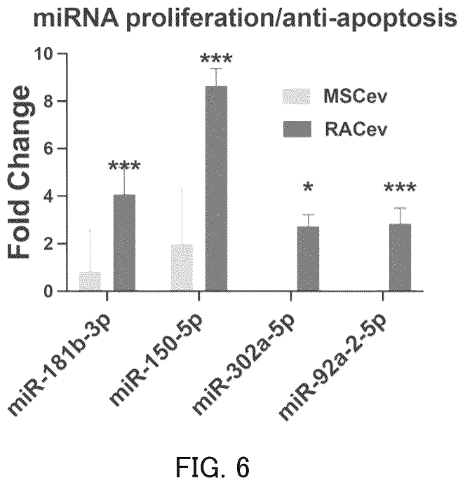

Next, FIG. 6 is a graph showing the expression level of miRNA. In FIG. 6, the horizontal axis indicates the type of miRNA, and the vertical axis indicates the relative expression level. As shown in FIG. 6, RACev had significantly high expression levels of miRNA (miR-92a-2-5p) having anti-apoptotic activity, miRNAs (miR-181b-3p, miR-150-5p, and miR-302a-5p) having cell division promoting activity, and miRNA (miR-150-5p) having cell migration promoting activity as compared to those of MSCev. From these results, it was found that RACev exhibits the vascular endothelial cell proliferation promoting activity by delivering miRNA having anti-apoptotic activity and miRNA having cell division promoting activity.

### (3) Suppression of Transplant Rejection

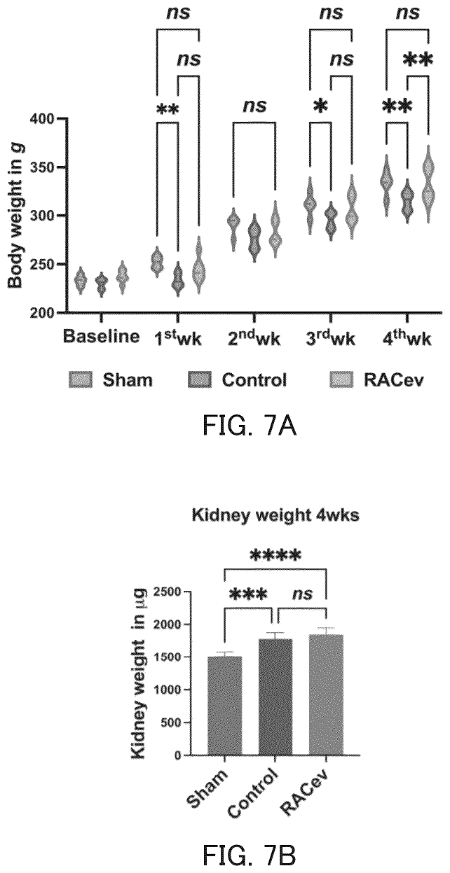

FIGS. 7A and 7B are graphs showing changes in body weight and organ weight (kidney weight) of the subject animals after 4 weeks. FIG. 7A is a graph showing changes in the body weights of the subject animals after 4 weeks, and FIG. 7B is a graph comparing the kidney weights of the subject animals after 4 weeks. In FIG. 7A, the horizontal axis indicates the elapsed time, and the vertical axis indicates the body weight (g). In FIG. 7B, the horizontal axis indicates the type of sample, and the vertical axis indicates the kidney weight (ug). In FIG. 7A, weight gain after 4 weeks was significantly increased in the RACev-treated group compared to the control group. In FIG. 7B, kidney weight after 4 weeks was increased in both of the control group and RACev-treated group compared to the Sham group, and they were almost equivalent. As shown in FIGS. 7A and 7B, no transplant rejection was observed by RACev transplantation. Statistical significance was determined by two-way analysis of variance with Tukey's multiple comparison test for body weight, and by one-way analysis of variance with Tukey's multiple comparison test for kidney weight. Results are shown as mean value ±SEM.

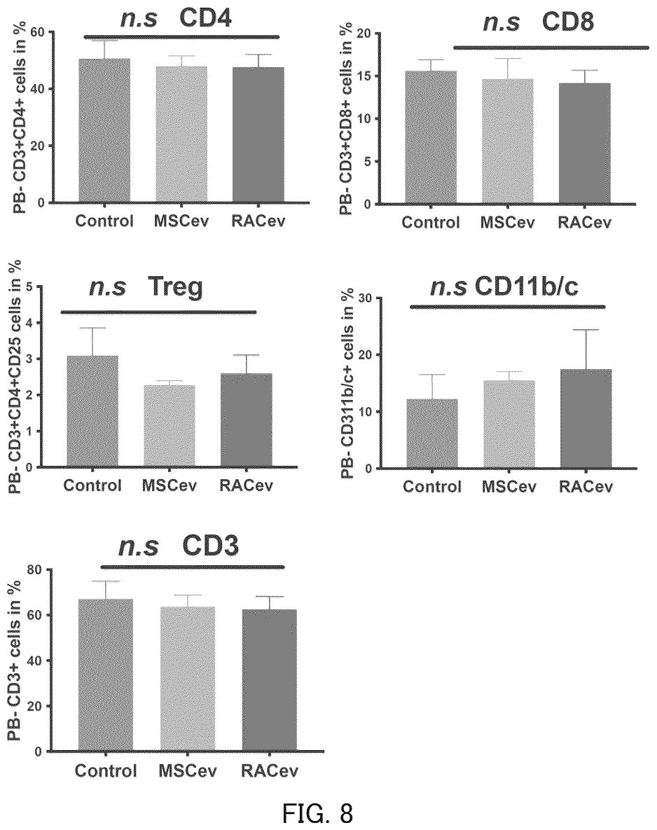

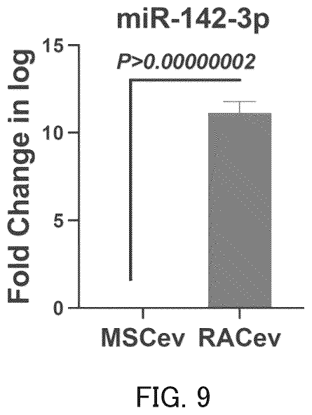

FIG. 8 shows graphs showing the proportions of CD3 positive cell, CD4 positive cell, CD8 positive cell, regulatory T cell, and CD11b/c positive cell in peripheral blood. In FIG. 8, the horizontal axis indicates the type of sample, and the vertical axis indicates the proportion of each cell. In FIG. 9, the horizontal axis indicates the type of sample, and the vertical axis indicates the relative expression level.

As shown in FIG. 8, there was no difference among RACev, MSCev and control in either peripheral blood or kidneys, and no transplant rejection was observed.

As shown in FIG. 9, RACev had a significantly high expression level of miRNA (miR-142-3p) having immunosuppressive activity (anti-inflammatory properties) as compared to that of MSCev. It is known that miR-142-3p is contained in exosomes released by regulatory T cells (Treg). From these results, it was suggested that RACev suppressed transplant rejection by delivering miRNA having immunosuppressive activity.

As shown in FIGS. 7A to 9, since no transplant rejection was observed, it was found that the composition of the present disclosure had an immunosuppression effect. Furthermore, the characteristics of immune cells in peripheral blood and kidney were clarified.

### (4) Improvement of Renal Function by RACev Transplantation

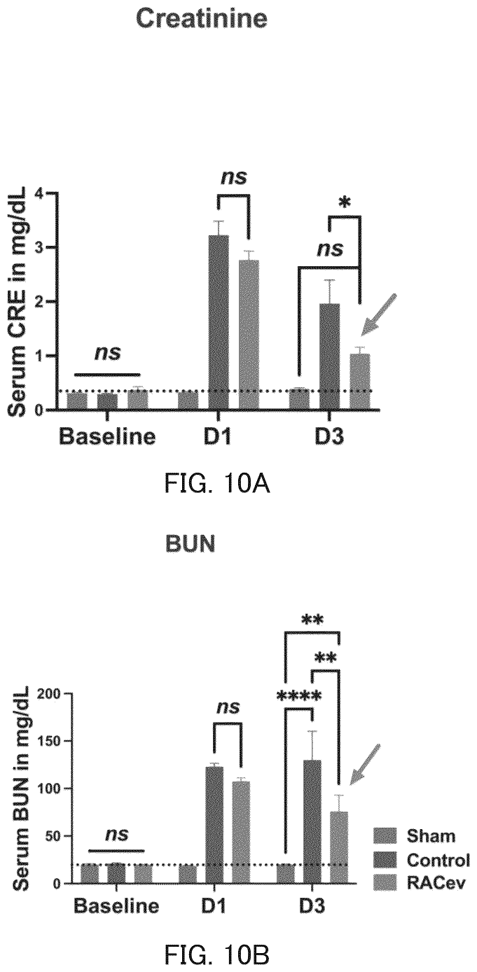

FIGS. 10A and 10B are graphs showing the serum creatinine (Cr) values and BUN values. FIG. 10A is a graph comparing changes in serum creatinine (Cr) values over time, and FIG. 10B is a graph comparing changes in serum BUN values over time. In FIG. 10A, the horizontal axis indicates the elapsed time, and the vertical axis indicates the serum creatinine (Cr) value (mg/dL). In FIG. 10B, the horizontal axis indicates the elapsed time, and the vertical axis indicates the serum BUN value (mg/dL).

On day 1 after the onset of acute liver dysfunction (R-AKI), EVs derived from 1×106 cells were injected via the tail vein, and the treatment duration was adjusted based on the clinical treatment protocol. In FIG. 10A, the serum creatinine (Cr) value on day 3 after the onset of K-IRI was significantly decreased in the EV-treated group compared to the control group (P<0.007, treatment group vs. control group). In FIG. 10B, serum BUN values were also markedly decreased in the RACev group compared to the control group (P<0.007, treatment group vs. control group). As shown in FIGS. 10A and 10B, improvement in renal function was observed with RACev transplantation. Statistical significance was determined by two-way analysis of variance with Tukey's multiple comparison test. Results are presented as mean value±SEM.

The body weight (BW) of the rats was measured before the onset and every week after the onset. Four weeks after the onset, the BW of the RACev-transplanted group increased compared to that of the control group (RACev; 333±6 g, control; 315±5.5 g, P<0.0053).

### (5) Improvement of Fibrosis

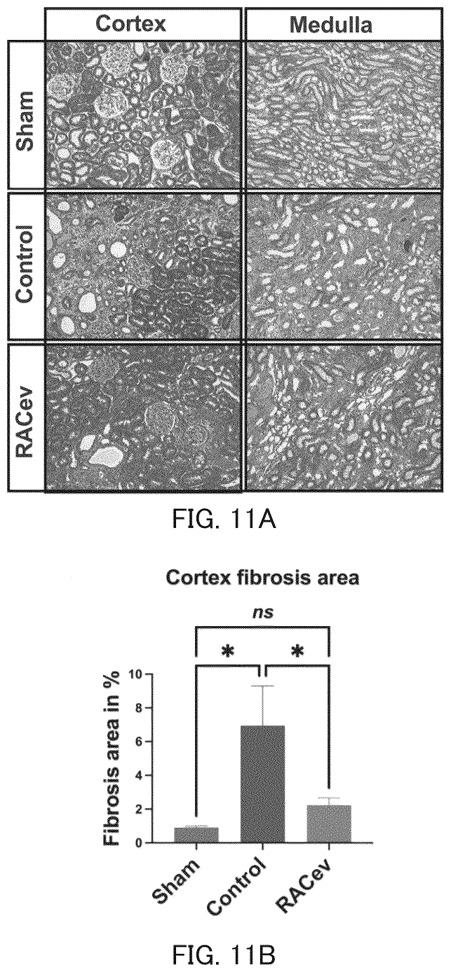

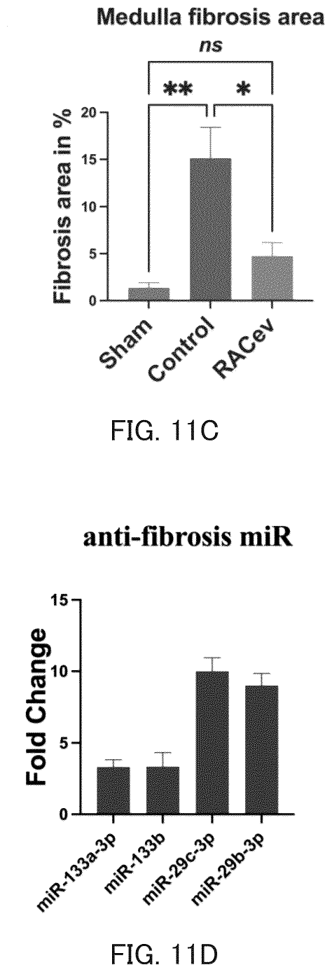

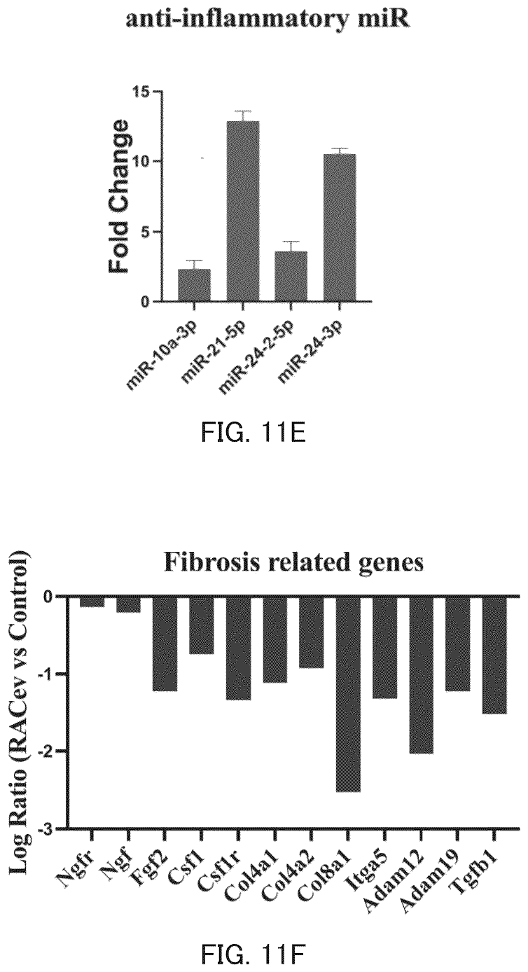

FIGS. 11A to 11F are photographs and graphs showing the degree of fibrosis. FIG. 11A shows fluorescence microscope images showing the degree of fibrosis in each part, FIG. 11B is a graph comparing the fibrosis areas in the cortex, FIG. 11C is a graph comparing the fibrosis areas in the medulla, FIG. 11D shows the expression levels of miRNAs with anti-fibrotic activity, FIG. 11E shows the expression levels of miRNAs with anti-inflammatory activity, and FIG. 11F shows expression levels of fibrosis-promoting genes. In FIG. 11B, the horizontal axis indicates the type of sample and the vertical axis indicates the fibrosis area (%). In FIG. 11C, the horizontal axis indicates the type of sample and the vertical axis indicates the fibrosis area (%). In FIGS. 11D and 11E, the horizontal axis indicates the type of RNA, and the vertical axis indicates the relative expression level. In FIG. 11F, the horizontal axis indicates the type of genes, and the vertical axis indicates the Log ratio (RACev to Control). As shown in FIG. 11A to 11C, Masson's trichrome staining showed that the kidney fibrosis level was maintained in the treatment group compared to that in the control group in both the cortex area and the medulla area (P<0.04 in cortex area, treatment group vs. control group; P<0.01 in the medulla area, treatment group vs. control group). As shown in FIGS. 11D and 11E, the anti-fibrotic effect of RACev combined with the delivery of miRNA with anti-fibrotic and anti-inflammatory activity to the ischemic tissue provided beneficial protection from excessive inflammation and scar formation. Furthermore, as shown in FIG. 11F, gene expression analysis revealed that the expression of most fibrosis-promoting genes (NGFR, FGF2, CSFLR, COL4A1, COL8A1, TGFb1, etc.) was significantly decreased in the RACev-treated group compared to the control group on day 6 after the onset of R-IRI. Thus, RACev transplantation increased renal function parameters by delivering miRNAs with anti-fibrotic and anti-inflammatory activity. Statistical significance was determined by one-way analysis of variance with Dunn's multiple comparison test. Results are presented as mean±SEM (n=8 to 10 per group).

### (6) Angiogenesis Promotion Activity of Extracellular Vesicles

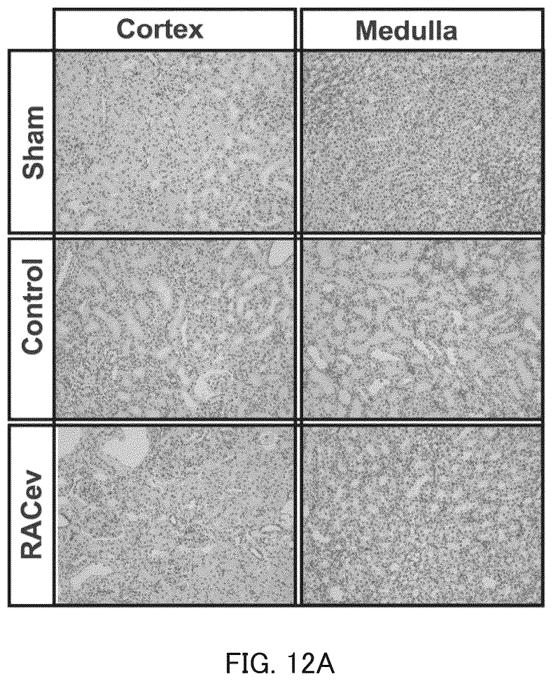

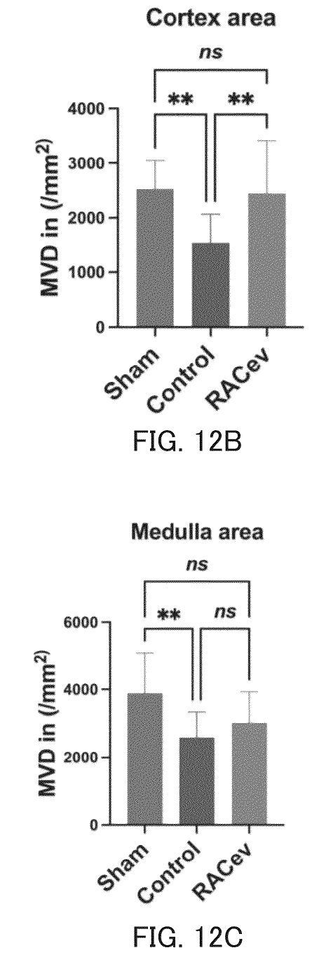

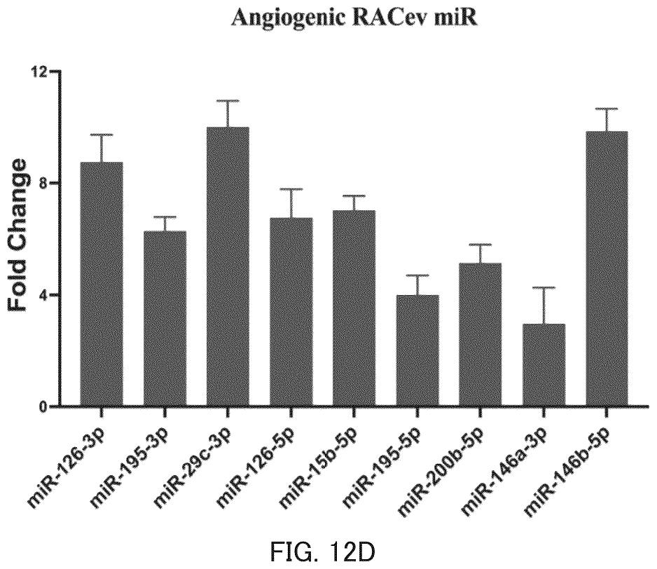

FIGS. 12A to 12D are photographs showing microvascular density in ischemic injured kidney tissue and graphs showing the number of CD31 positive microvessels. FIG. 12A shows blood vessels in ischemic injured kidney tissue, FIG. 12B is a graph comparing the vascular density in the cortex, FIG. 12C is a graph comparing the vascular density in the medulla, and FIG. 12D is a graph showing miRNA expression levels. In FIGS. 12B and 12C, the horizontal axis indicates the type of sample, and the vertical axis indicates microvascular density (/mm2). In FIG. 12D, the horizontal axis indicates the miRNA type, and the vertical axis indicates the relative expression level. FIGS. 12A to 12C show that microvascular density in ischemic injured kidney tissue was enhanced in the RACev-treated group (P<0.003, treatment group vs. control group), demonstrating potent angiogenic properties of RACev. In FIG. 12D, transcriptome analysis revealed that major angiogenic miRNAs involved in angiogenesis, so-called angiomiRs, are more highly up-regulated in RACev. Statistical significance was determined by one-way analysis of variance with Dunn's multiple comparison test. Results are presented as mean value±SEM (n=8 to 10 per group).

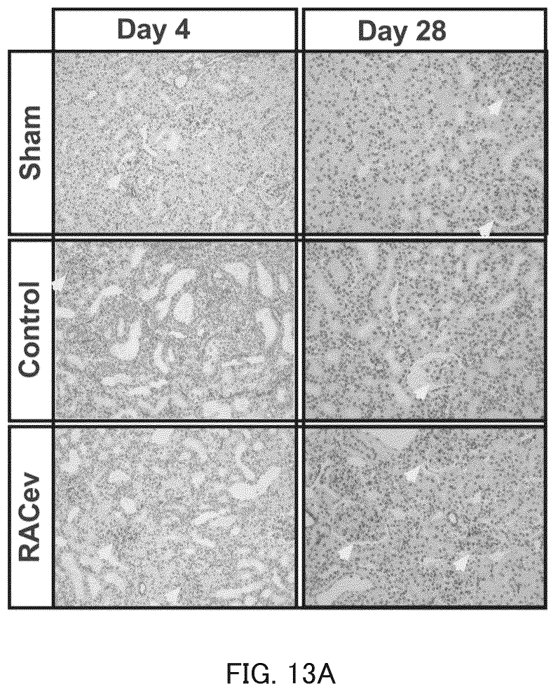

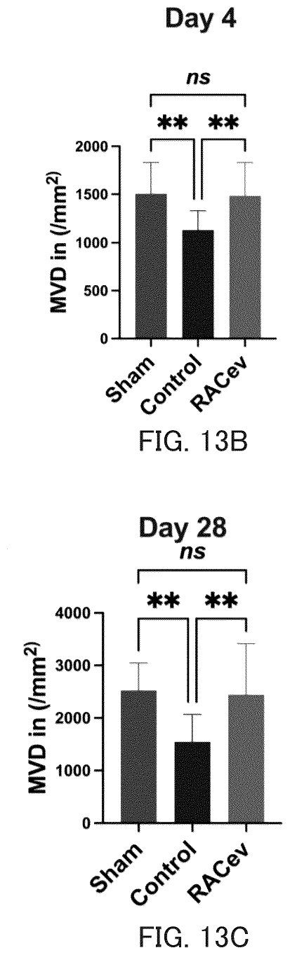

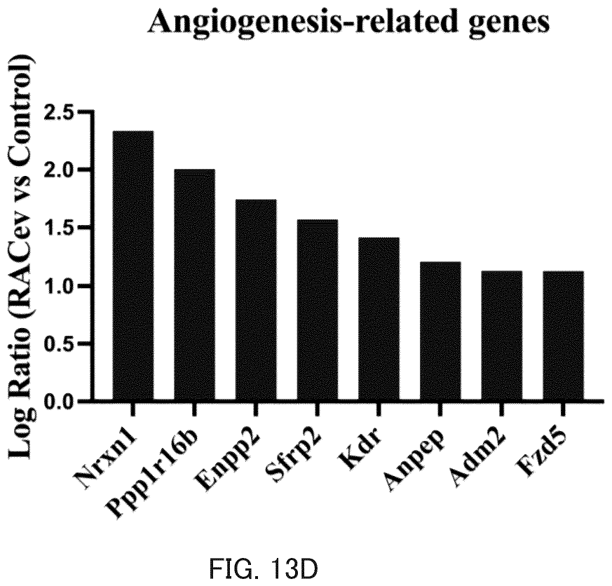

FIGS. 13A to 13D are photographs showing microvascular density in ischemic injured kidney tissue and graphs showing the number of CD31 positive microvessels on day 4 and day 28 after the onset of R-IRI. FIG. 13A shows blood vessels in ischemic injured kidney tissue, FIG. 13B is a graph comparing microvessel density in the cortex, FIG. 13C is a graph comparing microvessel density in the medulla, and FIG. 13D is a graph showing genes associated with angiogenesis. In FIGS. 13B and 13C, the horizontal axis indicates the type of sample, and the vertical axis indicates the microvascular density (/mm2). In FIG. 13D, the horizontal axis indicates the type of genes, and the vertical axis indicates the log ratio (RACev vs. Control). The capillary density was evaluated in the acute phase on day 6 and day 28 after the onset of R-IRI to verify its vascular tissue protective and angiogenic effects. FIGS. 13A and 13B show that microvascular density in ischemic injured kidney tissue was beneficially enhanced in the RACev-transplanted group compared to the control group (P<0.005 on day 6, RAC vs. control group, P<0.003 on day 28), demonstrating the potent vasculoprotective and angiogenic properties of RACev. Furthermore, the transcriptome analysis showed that RACev therapy significantly up-regulated angiogenesis-related genes in ischemic renal tissue compared to the control group (FIG. 13D). These results demonstrate that RACev delivers angiogenic enzymes and vasculoprotective miRs to promote angiogenesis and protect hypoxia-induced renal vascular network damage at the site of ischemic injury.

### (7) Selective RACev Accumulation in Damaged Kidneys

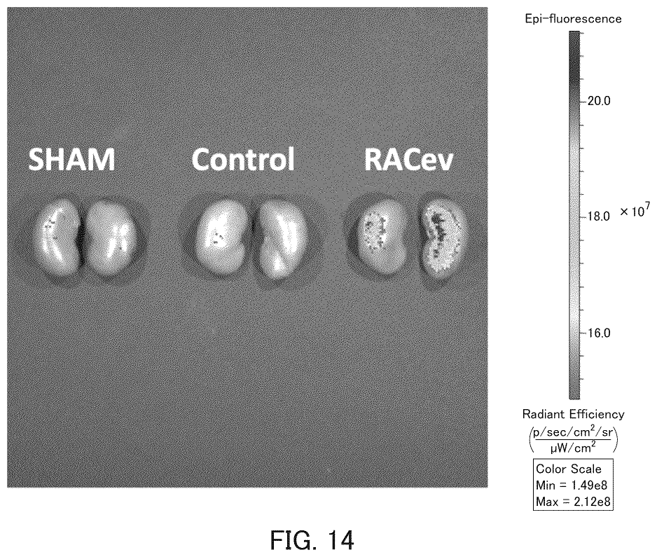

FIG. 14 is a photograph showing the results of IVIS measurement. As a result of IVIS measurement, it was verified that the labeled RACev accumulated in the damaged kidneys of the transplantation group. The above EV tracking analysis showed that RACev selectively accumulated in the liver.

The cell type annotation analysis was performed using the 500 most highly expressed genes to identify which nephron sites/cells showed high expression. The top 10 nephron regions with the highest expression levels were proximal tubular epithelial cell segments 1 and 2, distal tubular cell types 1 and 2, and renal tubular epithelial cells. This result confirms the IVIS result that most of the labeled RACev accumulated between the cortex and medulla. On the other hand, a part of the transplanted RACev was also captured in the kidneys. The above EV tracking analysis showed that RACev accumulated in the ischemia-induced renal tissue.

Under angiogenesis conditions, the inflammatory cell subset of peripheral blood mononuclear cells (PBMCs) changes its phenotype to regeneration-promoting cells such as angiogenic endothelial progenitor cells, M2 macrophages, and regulatory T cells. Notably, systemic injection of RACev significantly decreased serum creatinine and blood urea nitrogen on day 3 compared to the control group. Histologically, the treatment group had less fibrosis in the cortex area and the medulla area than the control group (P<0.04 and P<0.01). CD31 staining verified increased vascular density in the treatment group compared to that in the control group (P<0.003). These beneficial effects were associated with significant expression of miRNA involved in angiogenesis, anti-fibrosis, anti-inflammation, and anti-apoptosis. In vivo bioluminescence analysis showed that RACev preferentially accumulated in IR-injured kidneys. In conclusion, systemic transplantation of RACev improves renal function through protections such as suppression of tissue fibrosis, anti-inflammation, angiogenesis, and delivery of anti-apoptotic miRs to ischemic tissues.

### (8) Regulatory Roles of RACev on Epithelial-Mesenchymal Transition, Inflammation and Hypoxia After Ischemic Injury

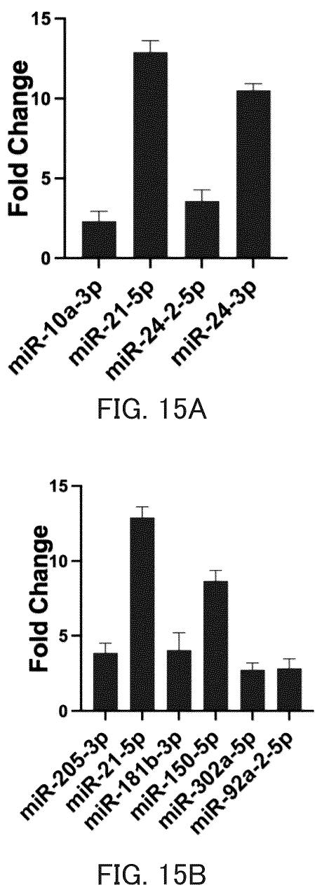

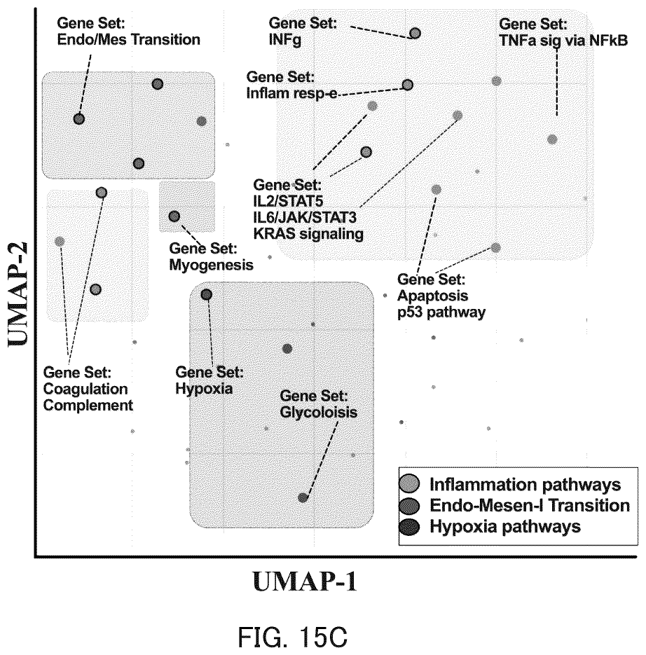

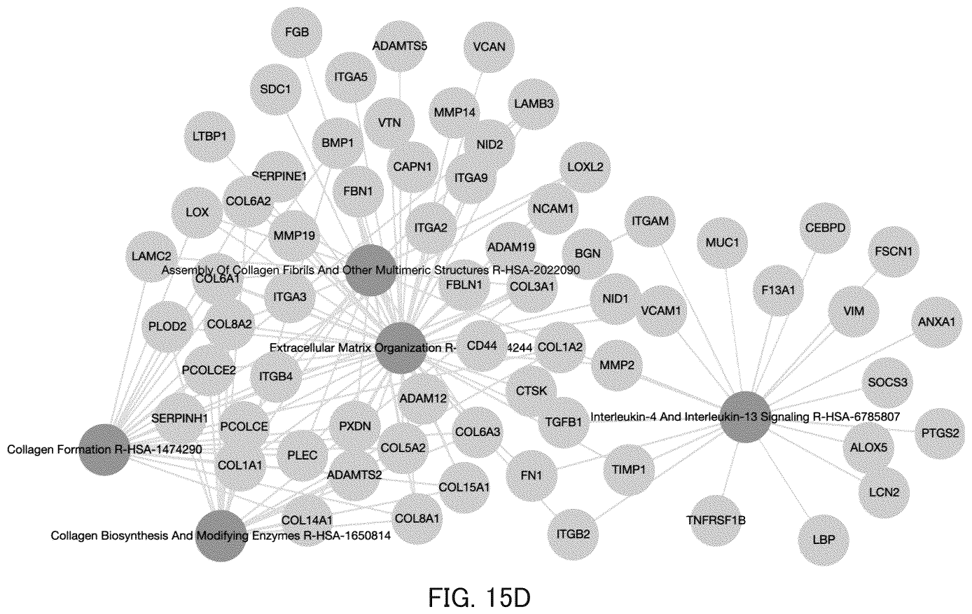

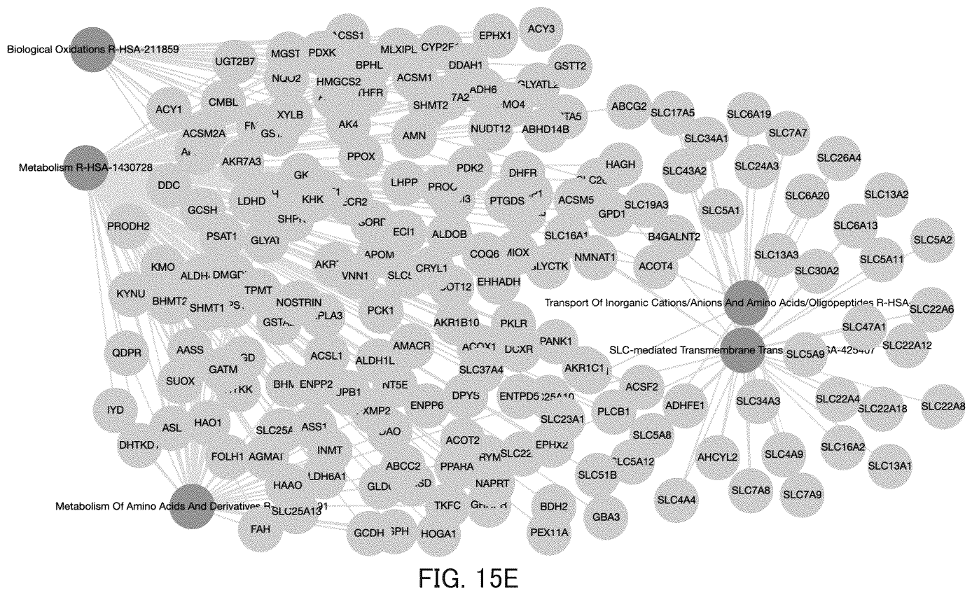

FIGS. 15A to 15E are graphs and diagrams showing the results of various analytical methods used to elucidate the mechanism of fibrosis reduction, demonstrating that renal fibrosis is significantly reduced 4 weeks after the onset of K-IRI following administration of RACev.

FIGS. 15A and 15B show miRNA expression levels, FIG. 15C shows cluster analysis results of the most relevant pathway regulation in the control group, FIG. 15D shows network analysis results of gene relationships in downstream mechanisms of the control group, and FIG. 15E shows network analysis results of gene relationships in downstream mechanisms of the treatment group. In FIGS. 15A and 15B, the horizontal axis indicates the type of RNA, and the vertical axis indicates the relative expression level. First, small non-coding RNA sequencing and data analysis revealed that RACev contains several important anti-inflammatory miRs, such as miR-10a-3p, miR-21-5p, miR-24-2-5p, and miR24-3p, which regulate excessive inflammation and initiate regeneration (FIG. 15A). Furthermore, RACev has anti-apoptosis and cell proliferation miRs (FIG. 15B). On the other hand, in the control group, pathway analysis showed that fibrosis of renal tissue was initiated by hypoxia pathway (P<2.19e-02) (FIGS. 15C and 15D), and the up-regulation of excessive inflammatory pathways such as IL-6-JAK2-STAT3 (P<1.43e-03), TNFα signaling via NF-KB (P<1.51e-03), and IFNγ response (P<2.19e-02) initiated the early epithelial-mesenchymal transition pathway (P<3.75e-11) (FIGS. 15C and 15D). In the treatment group, pathway analysis showed that RACev enhanced regeneration-related pathways (FIG. 15E).

While the present disclosure has been described above with reference to illustrative embodiments and examples, the present disclosure is by no means limited thereto. Various changes and modifications that may become apparent to those skilled in the art may be made in the configuration and specifics of the present disclosure without departing from the scope of the present disclosure.

This application claims priority from Japanese Patent Application No. 2021-181301 filed on Nov. 11, 2022. The entire subject matter of the Japanese Patent Application is incorporated herein by reference.

### SUPPLEMENTARY NOTES

Some or all of the above-described embodiments and examples may be described as the following supplementary notes, but they are not limited thereto.

### Supplementary Note 1

A composition for use in treating at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, the composition including: an extracellular vesicle of a regenerative cell population induced from a biological sample-derived mononuclear cell or a culture thereof.

### Supplementary Note 2

The composition according to Supplementary Note 1, wherein
the extracellular vesicle includes at least one miRNA selected from the group consisting of miR-15b-5p, miR-29b-3p, miR-29c-3p, miR-92a-2-5p, miR-126-3p, miR-126-5p, miR-133a-3p, miR-133b, miR-146a-3p, miR-146b-5p, miR-150-5p, miR-181b-3p, miR-195-3p, miR-195-5p, miR-200b-5p, miR-302a-5p, and miR-142-3p.

### Supplementary Note 3

The composition according to Supplementary Note 1 or 2, wherein
the extracellular vesicle further includes at least one miRNA selected from the group consisting of miR-10a-3p, miR-17-3p, miR-20b-5p, miR-21-5p, miR-24-2-5p, miR-29a-5p, miR-30b-5p, miR-30c-5p, miR-30e-3p, miR-30e-5p, miR-32-5p, miR-92a-3p, miR-103a-2-5p, miR-144-5p, miR-148a-3p, miR-199b-5p, miR-200a-3p, miR-205-5p, miR-210-3p, miR-221-5p, miR-324-5p, miR-363-3p, miR-373-3p, miR-509-3p, miR-633, and let-7c-5p.

### Supplementary Note 4

The composition according to any one of Supplementary Notes 1 to 3, wherein
the extracellular vesicle is positive for at least one extracellular vesicle marker selected from the group consisting of CD9 and CD63.

### Supplementary Note 5

The composition according to any one of Supplementary Notes 1 to 4, wherein
a mean diameter of the extracellular vesicle is in a range from 10 to 500 nm.

### Supplementary Note 6

The composition according to any one of Supplementary Notes 1 to 5, wherein
the composition has a vascular endothelial cell proliferation promoting activity.

### Supplementary Note 7

The composition according to any one of Supplementary Notes 1 to 6, wherein
the composition has an angiogenesis promoting activity.

### Supplementary Note 8

The composition according to any one of Supplementary Notes 1 to 7, wherein
the composition has a fibrosis suppressing activity.

### Supplementary Note 9

The composition according to any one of Supplementary Notes 1 to 8, wherein
the composition has a bioactivity with suppressed transplant rejection.

### Supplementary Note 10

The composition according to any one of Supplementary Notes 1 to 9, wherein
the extracellular vesicle is an extracellular vesicle extracted or isolated from the regenerative cell population or the culture thereof.

### Supplementary Note 11

The composition according to any one of Supplementary Notes 1 to 10, wherein
the regenerative cell population is induced by culturing the mononuclear cells in the presence of at least one factor selected from the group consisting of a stem cell factor, interleukin-6, FMS like tyrosine kinase-3 ligand, thrombopoietin, and a vascular endothelial cell growth factor.

### supplementary Note 12

The composition according to any one of Supplementary Notes 1 to 11, wherein
the regenerative cell population is induced without sorting at least one cell selected from the group consisting of a CD34 positive cell and a CD133 positive cell.

The composition according to any one of Supplementary Notes 1 to 12, wherein
the regenerative cell population includes endothelial progenitor cells, anti-inflammatory macrophages, and regulatory T cells.

### Supplementary Note 14

The composition according to Supplementary Note 13, wherein
the endothelial progenitor cell is a differentiated EPC colony-forming cell.

### Supplementary Note 15

The composition according to Supplementary Note 13 or 14, wherein
the anti-inflammatory macrophage is a M2 macrophage.

### Supplementary Note 16

The composition according to any one of Supplementary Notes 1 to 15, wherein
the biological sample is at least one tissue selected from the group consisting of blood and bone marrow.

### Supplementary Note 17

The composition according to any one of Supplementary Notes 1 to 16, wherein
the biological sample is at least one tissue selected from the group consisting of peripheral blood and umbilical cord blood.

### Composition for Treating at Least One Disease Selected From the Group Consisting of Kidney Diseases, Peripheral Arterial Diseases (PAD), Cerebral Infarction, Pulmonary Hypertension

### (PHD), Spinal Cord Ischemia, Non-Occlusive Mesenteric Ischemia, Ocular Ischemic Syndrome, Fibrosis, and Inflammatory Diseases

### Supplementary Note 18

A composition for use in therapeutic treatment or preventive treatment of at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, wherein
the composition is the composition according to any one of Supplementary Notes 1 to 17.

### Supplementary Note 19

The composition according to Supplementary Note 18, wherein the kidney disease is at least one disease selected from the group consisting of acute kidney injury, transitional phase chronic kidney diseases, chronic kidney diseases, and immunosuppression after kidney transplantation.

### Supplementary Note 20

A kidney disease pharmaceutical for use in therapeutic treatment or preventive treatment of at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, the kidney disease pharmaceutical including:
the composition according to any one of Supplementary Notes 1 to 17.

### Composition for Promoting Vascular Endothelial Cell Proliferation

### Supplementary Note 21

A composition for use in promoting vascular endothelial cell proliferation, wherein the composition is the composition according to any one of Supplementary Notes 1 to 17.

### Composition for Inducing Angiogenesis

### Supplementary Note 22

A composition for use in inducing angiogenesis, wherein the composition is the composition according to any one of Supplementary Notes 1 to 17.

### Composition for Suppressing Fibrosis

### Supplementary Note 23

A composition for use in suppressing fibrosis, wherein
the composition is the composition according to any one of Supplementary Notes 1 to 17.

### Method for Treating Kidney Disease

### Supplementary Note 24

A method for treating a patient with at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, the composition including:
administering to the kidney disease patient the composition according to any one of Supplementary Notes 1 to 17.

### supplementary Note 25

The method according to Supplementary Note 24, wherein the kidney disease is at least one disease selected from the group consisting of acute kidney injury, transitional phase chronic kidney diseases, chronic kidney diseases, and immunosuppression after kidney transplantation.

### Method for Promoting Vascular Endothelial Cell Proliferation

### Supplementary Note 26

A method for promoting vascular endothelial cell proliferation, including:
promoting vascular endothelial cell proliferation by bringing vascular endothelial cells into contact with a composition, wherein
the composition is the composition according to any one of Supplementary Notes 1 to 17.

### Method for Inducing Angiogenesis

### Supplementary Note 27

A method for inducing angiogenesis, the method using the composition according to any one of Supplementary Notes 1 to 17.

### Supplementary Note 28

The method according to Supplementary Note 27, including:
administering the composition to a subject to be induced with angiogenesis.

### Supplementary Note 29

The method according to Supplementary Note 27 or 28, wherein
the subject to be induced with angiogenesis is a patient with at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, and
the induction of angiogenesis is induction of angiogenesis in the kidney, brain, lung, bone marrow, intestine, eye, ischemic muscle, or at a site with ischemia-reperfusion injury of the patient.

### Method for Suppressing Fibrosis

### Supplementary Note 30

A method for suppressing fibrosis, the method using the composition according to any one of Supplementary Notes 1 to 17.

### Supplementary Note 31

The method according to Supplementary Note 30, including:
administering the composition to a subject for fibrosis suppression.

### Supplementary Note 32

The method according to Supplementary Note 30 or 31, wherein
the subject for fibrosis suppression is a patient with at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, and
the suppression of fibrosis is suppression of fibrosis in the kidney, brain, lung, bone marrow, intestine, eye, ischemic muscle, or at a site with ischemia-reperfusion injury of the patient.

### Use

### Supplementary Note 33

A composition for use in therapeutic treatment or preventive treatment of at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases, wherein the composition is the composition according to any one of Supplementary Notes 1 to 17.

### Supplementary Note 34

A composition for use in promoting vascular endothelial cell proliferation, wherein
the composition is the composition according to any one of Supplementary Notes 1 to 17.

### Supplementary Note 35

A composition for use in inducing angiogenesis, wherein
the composition is the composition according to any one of Supplementary Notes 1 to 17.

### supplementary Note 36

A composition for use in suppressing fibrosis, wherein
the composition is the composition according to any one of Supplementary Notes 1 to 17.

### INDUSTRIAL APPLICABILITY

As described above, since the composition of the present disclosure has suppressed transplant rejection and is derived from the regenerative cell population, the composition of the present disclosure has bioactivities, such as vascular endothelial cell proliferation promoting activity and angiogenesis promoting activity. Thus, the composition of the present disclosure can be suitably used, for example, for the treatment or prognosis improvement of at least one disease selected from the group consisting of kidney diseases, peripheral arterial diseases (PAD), cerebral infarction, pulmonary hypertension (PHD), spinal cord ischemia, non-occlusive mesenteric ischemia, ocular ischemic syndrome, fibrosis, and inflammatory diseases. Therefore, the present disclosure is extremely useful, for example, in the pharmaceutical field and the like.

1. A composition, comprising:
an effective amount of an extracellular vesicle of a regenerative cell population induced from a biological sample-derived mononuclear cell or culture thereof; and
a pharmaceutically acceptable carrier,
wherein the composition is effective for treating at least one disease selected from the group consisting of a kidney disease, a peripheral arterial disease (PAD), a cerebral infarction, pulmonary hypertension (PHD), spinal cord ischemia, non-occlusive mesenteric ischemia, ocular ischemic syndrome, fibrosis, and an inflammatory disease.

2. The composition according to claim 1, wherein
the extracellular vesicle comprises at least one miRNA selected from the group consisting of miR-15b-5p, miR-29b-3p, miR-29c-3p, miR-92a-2-5p, miR-126-3p, miR-126-5p, miR-133a-3p, miR-133b, miR-146a-3p, miR-146b-5p, miR-150-5p, miR-181b-3p, miR-195-3p, miR-195-5p, miR-200b-5p, miR-302a-5p, and miR-142-3p.

3. The composition according to claim 1, wherein
the extracellular vesicle comprises at least one miRNA selected from the group consisting of miR-10a-3p, miR-17-3p, miR-20b-5p, miR-21-5p, miR-24-2-5p, miR-29a-5p, miR-30b-5p, miR-30c-5p, miR-30e-3p, miR-30e-5p, miR-32-5p, miR-92a-3p, miR-103a-2-5p, miR-144-5p, miR-148a-3p, miR-199b-5p, miR-200a-3p, miR-205-5p, miR-210-3p, miR-221-5p, miR-324-5p, miR-363-3p, miR-373-3p, miR-509-3p, miR-633, and let-7c-5p.

4. The composition according to claim 1 3, wherein
the extracellular vesicle is positive for at least one extracellular vesicle marker selected from the group consisting of CD9 and CD63.

5. The composition according to claim 1, wherein
a mean diameter of the extracellular vesicle is in a range from 10 to 500 nm.

6. The composition according to claim 1, wherein
the extracellular vesicle exhibits a vascular endothelial cell proliferation promoting activity.

7. The composition according to claim 1, wherein
the extracellular vesicle exhibits an angiogenesis promoting activity.

8. The composition according to claim 1, wherein
the extracellular vesicle exhibits a fibrosis suppressing activity.

9. The composition according to claim 1, wherein
the extracellular vesicle exhibits a bioactivity with suppressed transplant rejection.

10. The composition according to claim 1, wherein
the extracellular vesicle is an extracellular vesicle extracted or isolated from the regenerative cell population or the culture thereof.

11. The composition according to claim 1, wherein
the regenerative cell population is induced by culturing the mononuclear cells in the presence of at least one factor selected from the group consisting of a stem cell factor, interleukin-6, FMS like tyrosine kinase-3 ligand, thrombopoietin, and a vascular endothelial cell growth factor.

12. The composition according to claim 1, wherein
the regenerative cell population is induced without sorting at least one cell selected from the group consisting of a CD34 positive cell and a CD133 positive cell.

13. The composition according to claim 1, wherein
the regenerative cell population comprises endothelial progenitor cells, anti-inflammatory macrophages, and regulatory T cells.

14. The composition according to claim 13, wherein
the endothelial progenitor cell is a differentiated EPC colony-forming cell.

15. The composition according to claim 13 wherein
the anti-inflammatory macrophage is a M2 macrophage.

16. The composition according to claim 1 wherein
the biological sample from which the mononuclear cell is derived is at least one tissue sample selected from the group consisting of blood and bone marrow.

17. The composition according to claim 1, wherein
the biological sample from which the mononuclear cell is derived is at least one tissue sample selected from the group consisting of peripheral blood and umbilical cord blood.

18. (canceled)

19. The composition according to claim 1, wherein
the kidney disease is at least one disease selected from the group consisting of acute kidney injury, transitional phase chronic kidney disease, chronic kidney disease, and immunosuppression following kidney transplantation.

20. (canceled)

21. (canceled)

22. (canceled)

23. (canceled)

24. The composition of claim 1, wherein the effective amount of the extracellular vesicle is in a range of 10⁴ to 10⁷ cells per kilogram of body weight per administration.

25. A method for treating a disease selected from the group consisting of a kidney disease, a peripheral arterial disease (PAD), cerebral infarction, pulmonary hypertension (PH), spinal cord ischemia, non-occlusive mesenteric ischemia, ocular ischemic syndrome, fibrosis, and an inflammatory disease in a subject in need thereof, the method comprising:
administering to the subject a therapeutically effective amount of a composition according to claim 1.
